(19) United States
(12) Patent Application Publication
(10) Pub. No.: US 2024/0238408 A1
(43) Pub. Date: Jul. 18, 2024

(54) CANCER IMMUNOTHERAPY BY DELIVERY OF mRNA

(71) Applicant: The University of Hong Kong, Hong Kong (CN)

(72) Inventors: Jiandong HUANG, Hong Kong (CN); Renhao LI, Hong Kong (CN)

(21) Appl. No.: 18/411,044

(22) Filed: Jan. 12, 2024

Related U.S. Application Data
(60) Provisional application No. 63/439,557, filed on Jan. 17, 2023.

(51) Int. Cl. A61K 39/215; A61K 39/00; A61P 35/00; C07K 16/28

(52) U.S. Cl. CPC A61K 39/215; A61P 35/00; C07K 16/2818; A61K 2039/505; A61K 2039/53; A61K 2039/6018

(57) ABSTRACT

This invention relates to cancer immunotherapy. A method to increase antigenicity or immunogenicity of a tumor in a subject is provided. In one embodiment, said method comprises the step of delivering lipid nanoparticles loaded with mRNA sequences encoding one or more pathogen antigen proteins into said tumor.

### FIELD OF THE INVENTION

The present invention generally relates to cancer immunotherapy, particularly, immunotherapeutic drugs.

### BACKGROUND OF THE INVENTION

In the past decades, cancer immunotherapy has been promising to improve cancer clinical therapy compared to traditional cancer therapy strategies. However, existing cancer immunotherapy cannot benefit all cancer patients with different cancer types and stages. Effective cancer immunotherapies always rely on the induction of the cancer immunity cycle [1]. In this cycle, tumor antigens are released by anti-tumor responses and captured by professional antigen-presenting cells (APCs). And then, APCs present the antigen on the MHC molecules to prime and active tumor-specific T cells. Finally, these activated tumor-specific T cells are recruited to tumors, recognize and kill their target cancer cells. The elimination of cancer cells results in the release of more tumor-associated antigens to induce more extensive and robust anti-tumor responses in the subsequent cycle [1]. Nevertheless, tumors constantly evolve to escape immunosurveillance or disrupt the cancer immunity cycle, which finally results in the failure of immunotherapy or the recurrence of the tumor [2, 3].

The three most important elements for better therapeutic efficacy in this cancer immunity cycle are tumor antigenicity, tumor immunogenicity, and the tumor microenvironment [3], which are for easy tumor recognition, potent tumor-specific immune response induction, and effective intratumoral immune cell activation.

One of the strategies to induce an effective cancer immunity cycle is introducing pathogens into the tumor to increase the immunogenicity of the tumor and reverse the suppressive tumor microenvironment. For example, one study showed that the intratumoral injection of the unadjuvanted seasonal influenza vaccine reduced tumor growth by converting the “cold” tumor microenvironment to a “hot” one [4]. Another potential cancer therapeutic approach is oncolytic viruses, which could selectively infect cancer tissue without normal tissue infection, so, this virus could be modified to deliver drugs or redirected for specific targeting. At the same time, the immunogenicity of the oncolytic virus also helps to convert the suppressive tumor microenvironment [5, 6]. Besides, bacteria-based cancer therapy is also becoming attractive, especially the genetically engineered tumor-targeting attenuated bacteria has shown robust tumor-killing efficacy and significant survival increase. It was previously shown that the introduction of engineered tumor-targeted Salmonella in vivo can efficiently suppress tumor growth [7, 8] and tumor metastasis [9].

On the other hand, to make the tumor can be recognized by the immune system easier, people increase the antigenicity and immunogenicity of the tumor by introducing foreign antigens into the tumor. One strategy is directly introducing foreign MHC into tumor cells to increase antigenicity and immunogenicity and generate systemic antitumor immune responses [10, 11]. Moreover, people transloaded the tumor cells' MHC with influenza virus-derived peptides to be used as vaccines [12], to help to induce more robust anti-tumor responses.

However, not only the pathogen-based or tumor-antigen-based cancer immunotherapies, but also the widely and clinically used strategies immune checkpoint blockade [13-16], CAR-T therapy [17-21], etc., focus only on a single aspect needed to eradicate tumors, i.e., either on tumor microenvironment conversion, or tumor recognition, which only have limited therapeutic efficacy when they are used alone. One strategy is to not only regulate the tumor microenvironment but also increase the recognition of the tumor by expressing the foreign antigen with enough immunogenicity and antigenicity to induce a potent tumor elimination rapidly.

BNT162b2 (Comirnaty) is an mRNA-based COVID-19 vaccine developed by BioNTech company. BNT162b2 is made up of Spike protein mRNA and lipid nanoparticles [22]. Studies have shown that BNT162b2 administration can effectively prevent SARS-COV-2 infection in humans and mice by provoking potent anti-Spike protein humoral and cellular immune responses [23-25]. Due to the COVID-19 pandemic, for now, more than 69% of the world population has received at least one dose of a COVID-19 vaccine [26], which has induced the memory immune responses targeting spike protein of SARS-COV-2 to prevent the infection or the severe symptoms. It is therefore an ideal candidate to demonstrate the general cancer therapy strategy of this invention.

In addition to BNT162b2 mRNA vaccine, encoding SARS-COV-2 Spike protein, lipid nanoparticle encapsulated mRNA vaccine encoding HKU1 coronavirus-Spike protein or staphylococcal enterotoxin A (SEA) superantigen were also developed for cancer therapy in this invention.

This invention showed that lipid nanoparticle encapsulated mRNA has great cancer therapy potential in various types of cancer by expressing spike protein in tumor cells and inducing potent anti-spike protein T cell immune responses. In addition, this first anti-tumor attack could induce potent tumor-antigen spreading by spike protein-contained tumor exosome release and dead tumor cells. Therefore, this invention provides a powerful cancer therapy strategy by repurposing the existing mRNA vaccines, such as COVID-19 mRNA vaccines, for cancer therapy with robust cancer therapeutic efficacy and rapid clinical translation potential for a number of cancers. In addition, this invention also showed other antigens from pathogens have great cancer therapy potential.

### SUMMARY OF THE INVENTION

This invention provides a method to increase antigenicity or immunogenicity of a tumor in a subject. In one embodiment, said method comprises the step of delivering lipid nanoparticles loaded with mRNA sequences encoding one or more pathogen antigen proteins into said tumor.

This invention also provides a method to treat a subject having one or more tumors, said method comprises increasing antigenicity or immunogenicity of at least one tumor among said one or more tumors using the method of this invention.

This invention further provides an intratumoral injection for treating tumor in a subject comprising lipid nanoparticles loaded with mRNA encoding one or more pathogen antigen proteins.

This invention also provides a kit for treating tumor in a subject, comprising the intratumoral injection of this invention.

This invention also provides a combinational therapy of this invention with immune checkpoint inhibitors.

### BRIEF DESCRIPTION OF THE DRAWINGS

FIG. 1 shows an embodiment of the therapeutic strategy of this invention.

FIG. 2 shows the plasmid gene map for B16F10-OVA cell line construction.

FIG. 3A shows the experimental design of BNT16262 cancer therapy. BNT162b2 intramuscular vaccinations were carried out before cancer cell implantation. n=5 for each group.

FIG. 3B shows tumor growth curves of the BNT162b2 intratumoral injection treatment group vs. the PBS intratumoral injection control group for the experiment described in FIG. 3A.

FIG. 3C shows tumor growth curves of PBS intratumoral injection control group for the experiment described in FIG. 3A.

FIG. 3D shows tumor growth curves of the BNT162b2 intratumoral injection treatment group for the experiment scheme described in FIG. 3A.

FIG. 3E shows tumor comparison photo of treatment and control group from the experiment described in FIG. 3A.

FIGS. 4A to 4V show intratumoral injection of BNT162b2 reduces tumor growth or tumor lung metastasis in vaccinated mice. BNT162b2 intramuscular vaccinations were carried out after cancer cell implantation.

FIGS. 4A, 4F, 4K, and 4R show the experimental design of BNT162b2 cancer therapy. BNT162b2 vaccinations were carried out after cancer cell implantation. n=5 or 6 in each group.

FIG. 4B shows B16F10 tumor growth curves of the BNT162b2 intratumoral injection treatment group vs. the PBS intratumoral injection control group for the experiment described in FIG. 4A.

FIG. 4C shows B16F10 tumor growth curves of PBS intratumoral injection control group for the experiment described in FIG. 4A.

FIG. 4D shows B16F10 tumor growth curves of the BNT162b2 intratumoral injection treatment group for the experiment scheme described in FIG. 4A.

FIG. 4E shows B16F10 tumor comparison photo of treatment and control group from the experiment described in FIG. 4A.

FIG. 4G shows MB49 tumor growth curves of BNT162b2 intratumoral injection treatment group vs. PBS intratumoral injection control group from the experiment described in FIG. 4F.

FIG. 4H shows MB49 tumor growth curves of PBS intratumoral injection control group of the experiment described in FIG. 4F.

FIG. 4I shows MB49 tumor growth curves of BNT162b2 intratumoral injection treatment group of the experiment described in FIG. 4F.

FIG. 4J shows MB49 tumor comparison photo of treatment vs. control group from the experiment scheme described in FIG. 4F.

FIG. 4K shows the experimental design of BNT162b2 cancer therapy. BNT162b2 vaccinations were carried out after 4T1 cancer cell implantation. n=6 in each group.

FIG. 4L shows 4T1 tumor growth curves of BNT162b2 intratumoral injection treatment group vs. PBS intratumoral injection control group from the experiment described in FIG. 4K.

FIG. 4M shows 4T1 tumor growth curves of PBS intratumoral injection control group of experiment described in FIG. 4K.

FIG. 4N shows 4T1 tumor growth curves of BNT162b2 intratumoral injection treatment group of the experiment described in FIG. 4K.

FIG. 4O shows 4T1 tumor comparison photo of treatment vs. control group from the experiment scheme described in FIG. 4K.

FIG. 4P shows 4T1 tumor lung metastasis comparison photo of treatment and control group at the endpoint of the experiment described in FIG. 4K.

FIG. 4Q shows the number of lung metastases per mouse for each group (n=6 per group) shown in FIG. 4P.

FIG. 4R shows the CT26 subcutaneous model of this invention. The tumor growth was monitored and measured every two days. BNT162b2 intramuscular vaccinations were carried out after cancer cell implantation.

FIGS. 4S to 4V show that the BNT162b2 therapy also can effectively inhibit the CT26 colon cancer tumor growth.

FIGS. 5A to 5F, and 5H to 5V show the intratumoral BNT162b2 vaccine injection induced potent anti-tumor T cell immune response.

FIGS. 5G-1, 5G-2, 5G-3 and 5G-4 showed the recruitment of immune cells in tumor.

FIGS. 5A and 5K show the experimental design.

FIGS. 5B, 5C, 5G-1, 5G-2, 5G-3 and 5G-4 show the detection of tumor-infiltrated immune cells by flow cytometry or immunofluorescent staining.

FIGS. 5D and SE show the detection of Spike protein expression on the tumor cell surface and intratumoral by immunofluorescence staining of tumor sections. BNT i.m.-BNT i.t: tumor section from mice with intramuscular and intratumoral BNT162b2 injection. BNT i.m.-PBS i.t: tumor section from mice with BNT intramuscular vaccination and intratumoral PBS injection. Tumor cell surface Spike, Intratumoral Spike, and DAPI are shown in the figures.

FIGS. 5F, 5G-1, 5G-2, 5G-3 and 5G-4 show the comparison of tumor-infiltrated T cells. B cells, NK cells, Antigen presenting cells, Macrophages, and Neutrophages in the BNT162b2 intratumoral injection therapy group and PBS intratumoral injection control group. FIGS. 5G-2 and 5G-4 showed the result of intratumoral immune cells staining by flow cytometry.

FIG. 5H shows the anti-Spike protein IgG antibody titer post intratumoral BNT162b2 injection therapy.

FIG. 5I shows representative IFN-γ ELISpot shows tumor-antigen-specific T cell responses post BNT intratumoral injection therapy.

FIG. 5J shows the bar graph showing the summary of ELISpot data, comparing the tumor-antigen specific T cell responses in BNT162b2 intratumoral injection treatment group and PBS intratumoral injection control group. Tumor cell line lysis was used as tumor-antigen stimulator. X-PBS i.t: splenocytes from mice without any intramuscular vaccination but with intratumoral PBS injection. BNT i.m.-BNT i.t: splenocytes from mice with intramuscular and intratumoral BNT162b2 injection. BNT i.m.-PBS i.t: splenocytes from mice with BNT162b2 intramuscular vaccination and intratumoral PBS injection.

FIG. 5L shows tumor growth curves of the BNT16262 intratumoral injection treatment group vs. the PBS intratumoral injection control group for the experiment described in FIG. 5K.

FIG. 5M shows tumor comparison photo of treatment and control group from the experiment described in FIG. 5K.

FIG. 5N shows tumor growth curves of BNT162b2 intratumoral injection treatment group and PBS intratumoral injection control group in μMT mice for the experiment described in FIG. 5K.

FIG. 5O shows tumor growth curves of BNT162b2 intratumoral injection treatment group and PBS intratumoral injection control group in Rag1^{-/-} mice for the experiment described in FIG. 5K.

FIG. 5P Experimental design of the detection of memory T cell activation post-BNT162b2 intratumoral treatment.

FIG. 5Q shows the CD69+ activated T cell detection at the timepoint described in FIG. 5P.

FIG. 5R shows the IFN-γ+ activated T cell detection at the timepoint described in FIG. 5P.

FIG. 5S shows the schematic diagram of the experimental design of the RNA-seq.

FIG. 5T shows the Venn plot comparing the differential gene expression between intratumoral BNT162b2 treatment group (BNT i.m.-BNT i.t. group) and the two control groups (BNT i.m.-PBS i.t. group and PBS i.t. only group) at different time points.

FIG. 5U shows the enriched GO terms of significantly upregulated genes after BNT162b2 intratumoral injections.

FIG. 5V shows the correlation between APC markers and T cell activation markers. Wilcoxon test was used to analyze statistical differences.

FIGS. 6A to 6D show the tumor microenvironment study post BNT162b2 intratumoral injections.

FIGS. 6A and 6B show the phenotypes of intratumoral macrophage in tumors post BNT162b2 treatments or PBS treatments.

FIGS. 6C-1 and 6C-2 show cytokine profiles in tumors or in circulations from the mice post BNT162b2 treatments or PBS treatments.

FIG. 6D shows MHC-I and MHC-II expression in tumors from the BNT162b2 therapy group and the control group.

FIGS. 7A to 7N show the mechanism investigation of antigen spreading induced by BNT162b2 cancer therapy.

FIG. 7A shows the experimental design for the investigation of exosomes induced by BNT162b2 treatment.

FIG. 7B shows the flow cytometry intracellular staining to compare the level of CD63+ exosome secretion post-BNT162b2 treatment to DMSO treatment.

FIG. 7C shows the immunofluorescent staining of intracellular CD63 to detect the level of exosome secretion in tumors post BNT162b2 intratumoral injections and PBS intratumoral injections.

FIG. 7D shows the experimental design of the investigation of exosomes of B16F10-OVA post BNT162b2 transfection.

FIG. 7E shows the transmission electron microscopy (TEM) image of isolated B16F10-OVA-derived exosome.

FIG. 7F shows the identification of B16F10-OVA-derived exosome by western blot.

FIG. 7G shows the experimental design for investigating the potential of antigen spreading induced by tumor cells derived exosomes.

FIG. 7H shows the ELISpot assay to detect the antigen spreading induced by tumor cells-derived exosomes.

FIG. 7I shows the experimental design of the investigation of heat shock protein secretion induced by BNT162b2 transfection in tumor cells.

FIG. 7J shows the statistical chart of the level of Calreticulin and HSP70 heat shock protein secretion post BNT162b2 transfection in tumor cells.

FIG. 7K shows the experimental design of the antigen spreading and the distal therapeutic efficacy detection in the bilateral tumor model.

FIG. 7L shows tumor photos and tumor growth curve of the experiment described in FIG. 7K.

FIG. 7M shows the experimental design of the potential of antigen spreading induced by dead tumor cells.

FIG. 7N shows the ELISpot assay to detect the immunogenicity of the dead tumor cells for antigen spreading.

FIG. 8A to 8N show the role of PD-L1 expression during the BNT162b2 cancer therapy.

FIG. 8A shows the experimental design for the BNT162b2 cancer therapy, and samples are obtained for immunodetection at the endpoint.

FIG. 8B shows the PD-L1 expression detection in the tumor by immunofluorescent staining.

FIG. 8C shows the detection of intratumoral PD-L1 expression on tumor cells and CD45+ Leukocytes by flow cytometry.

FIG. 8D shows the transcriptome analysis of PD-L1 expression in the tumor at different timepoints during BNT162b2 cancer therapy.

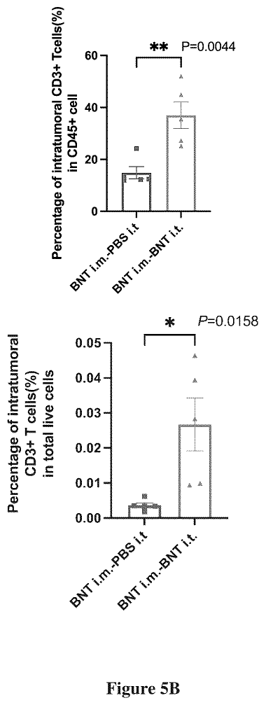

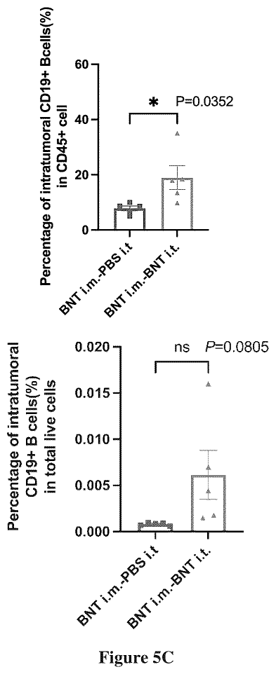

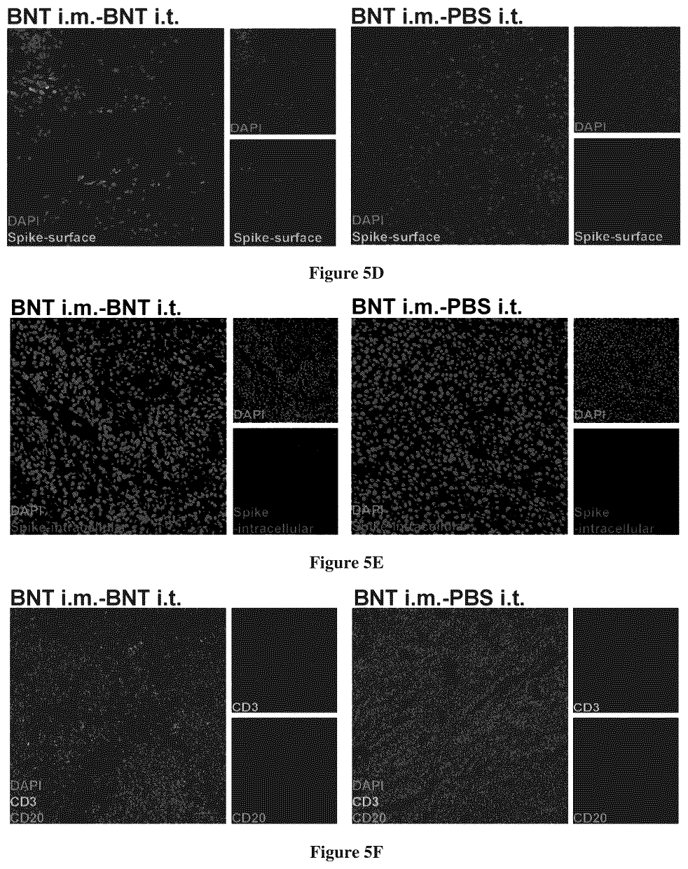

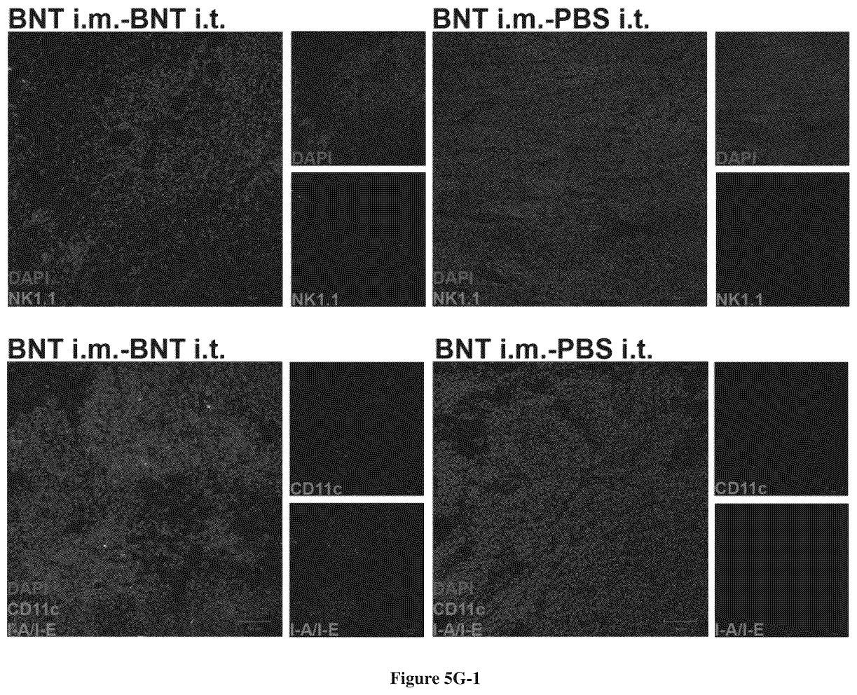

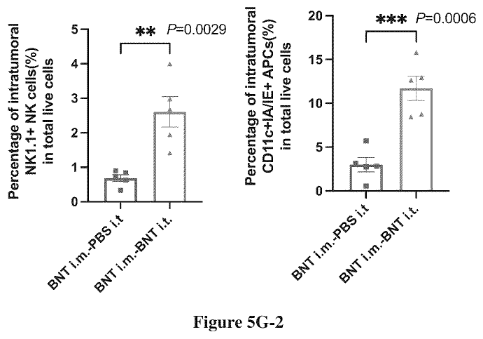

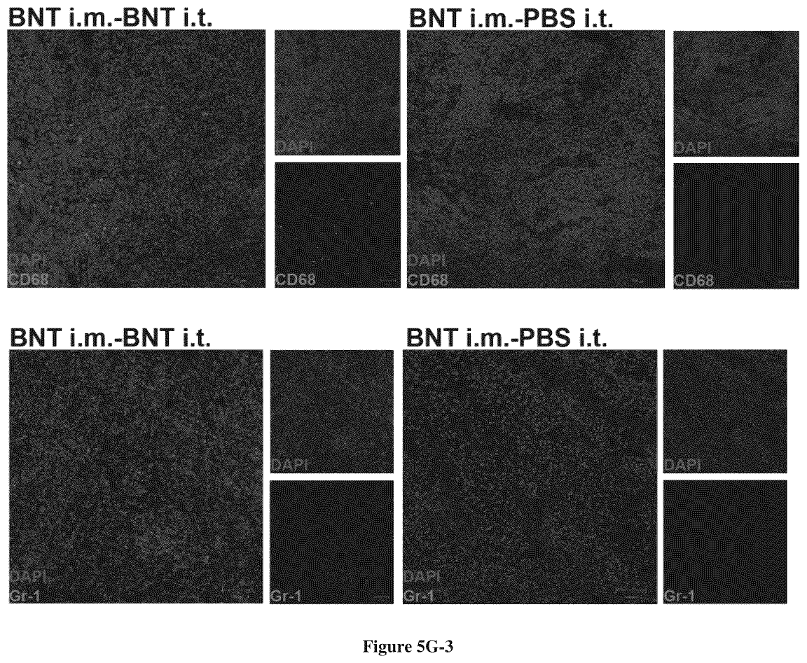

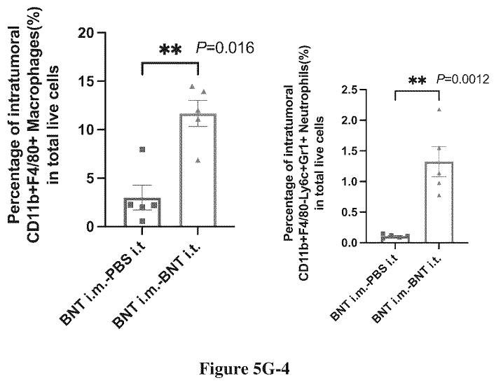

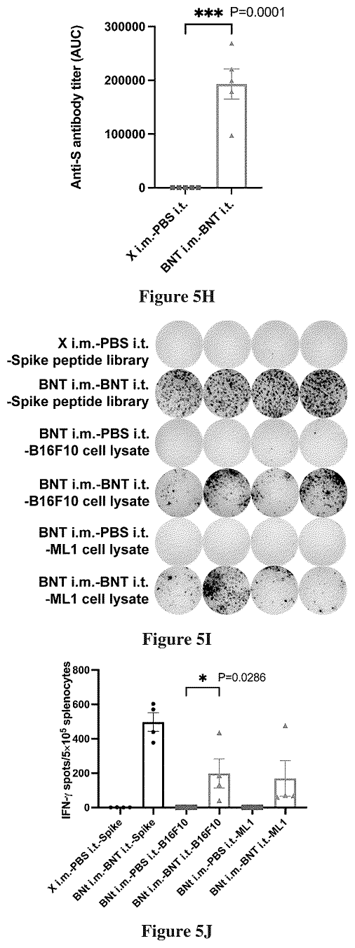

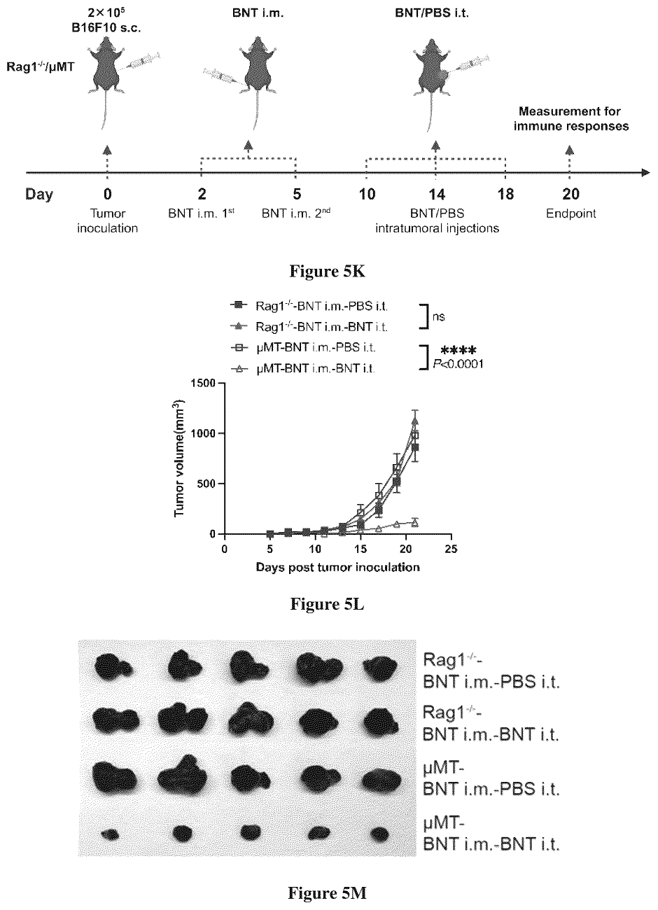

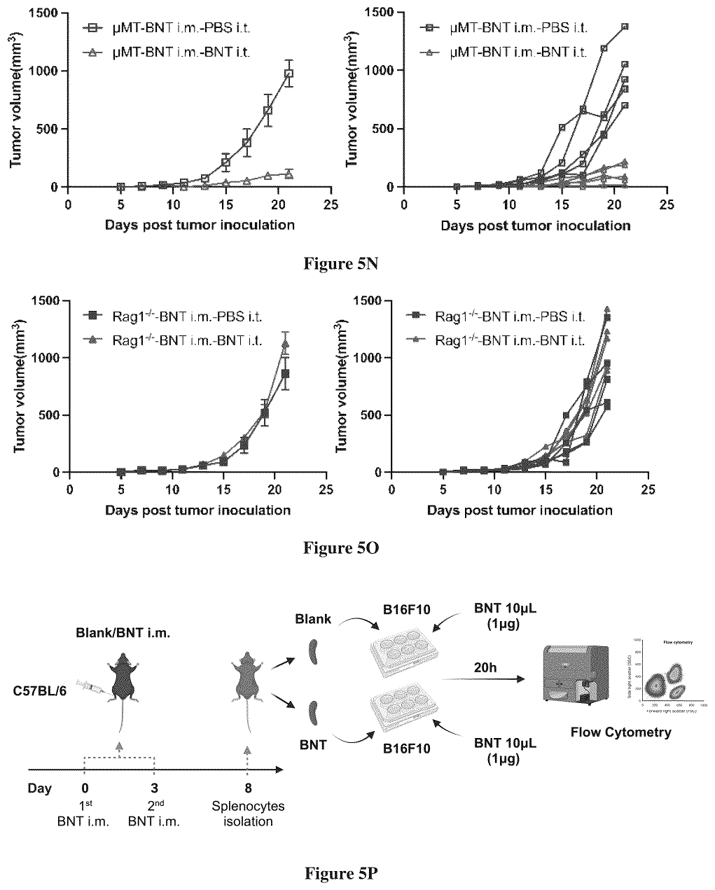

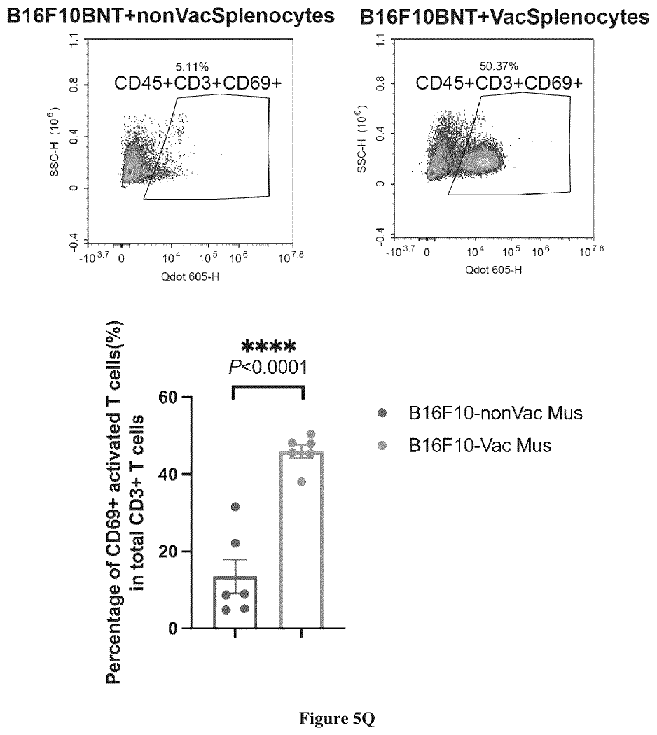

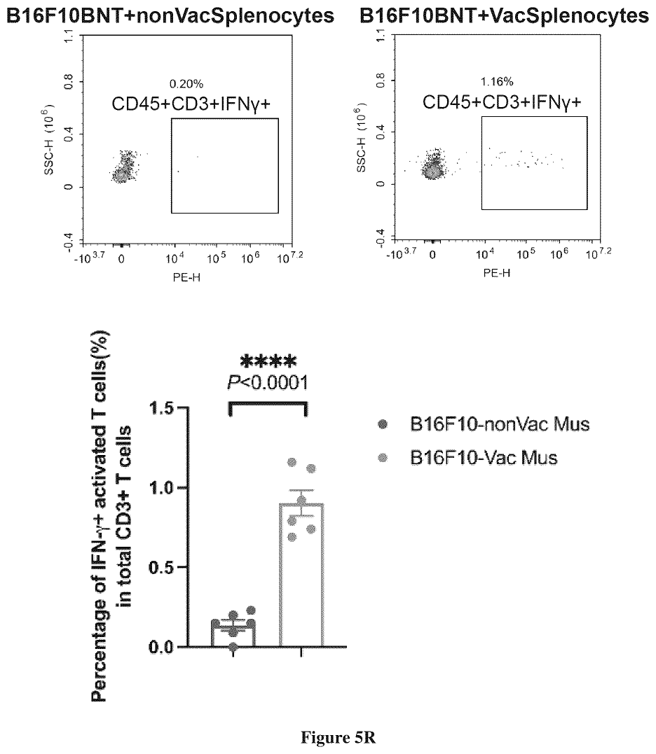

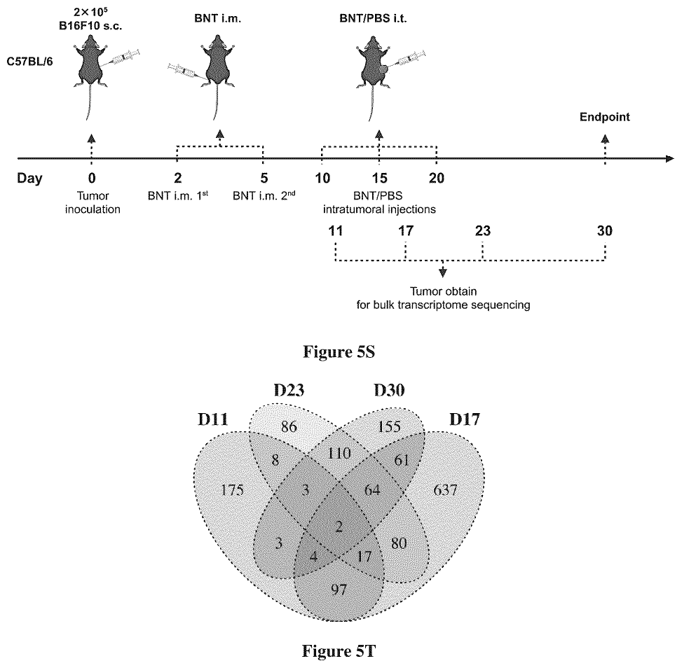

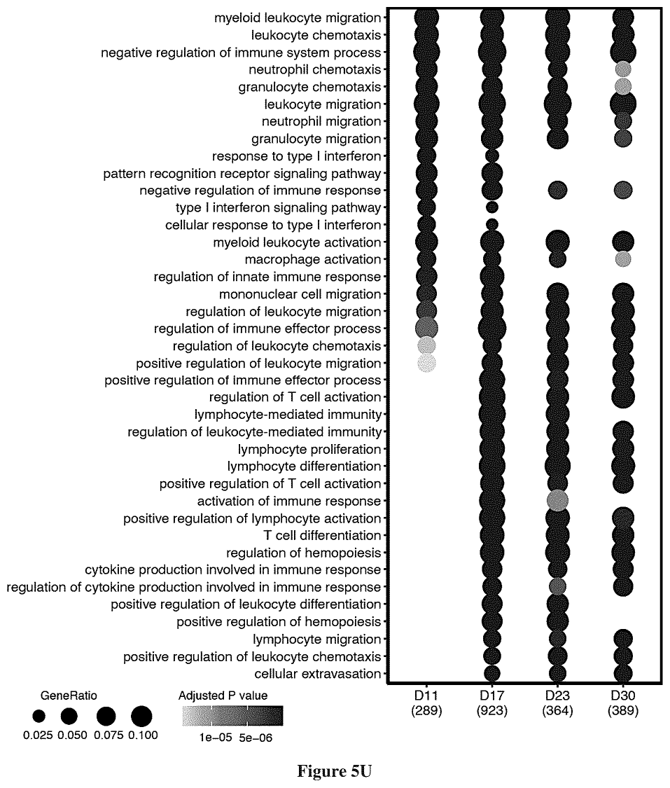

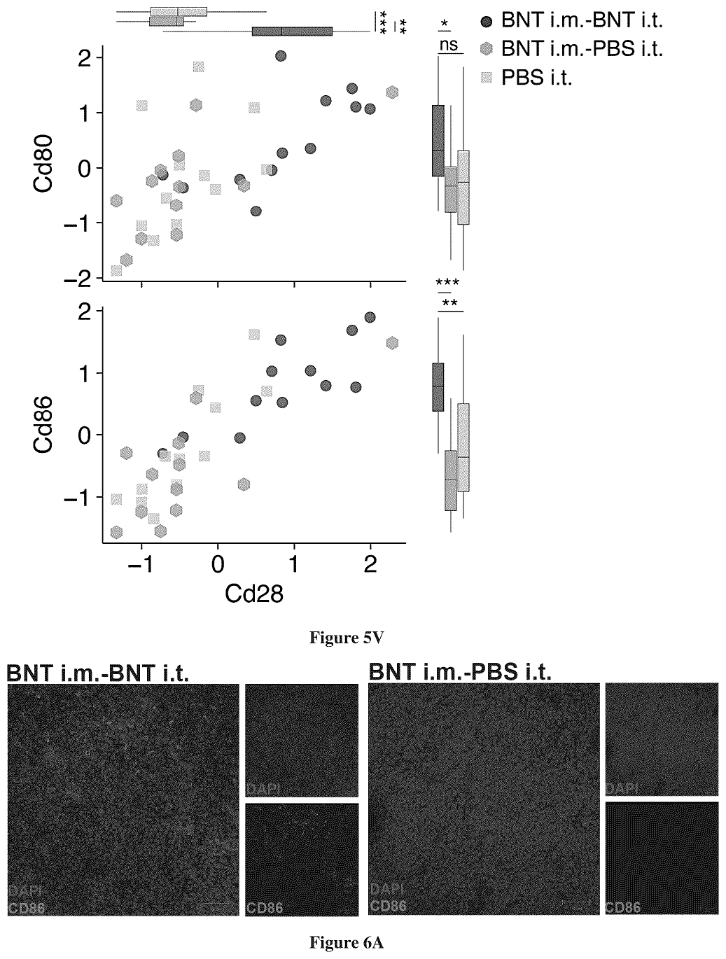

FIG. 5E shows the transcriptome analysis of the correlation between PD-L1 expression and cell types.

FIG. 5F Heatmap shows the fold change of neutrophil and T cell activation genes after BNT162b2 intratumoral injections.

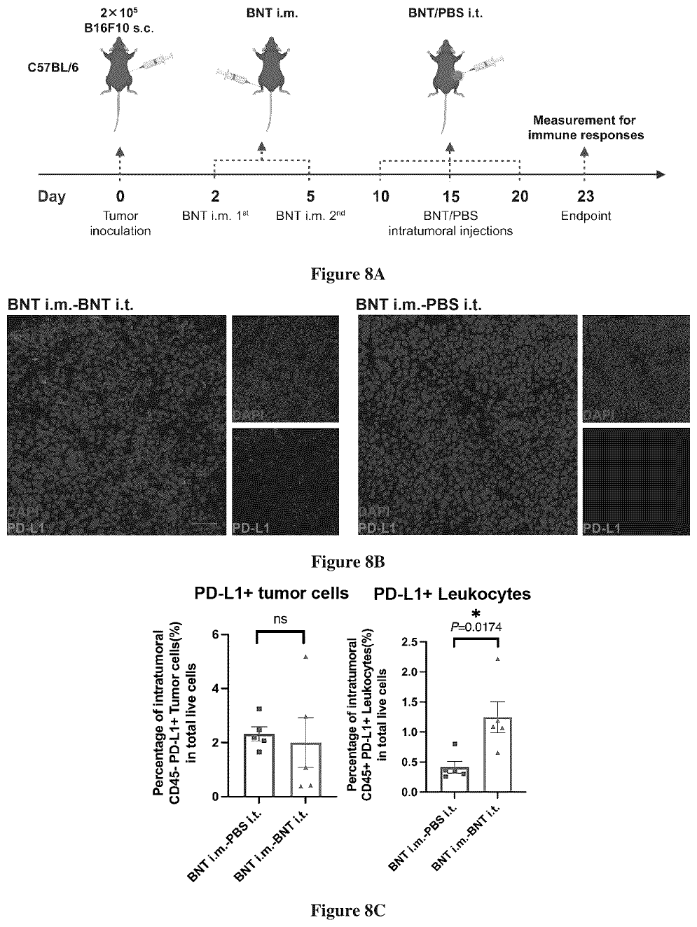

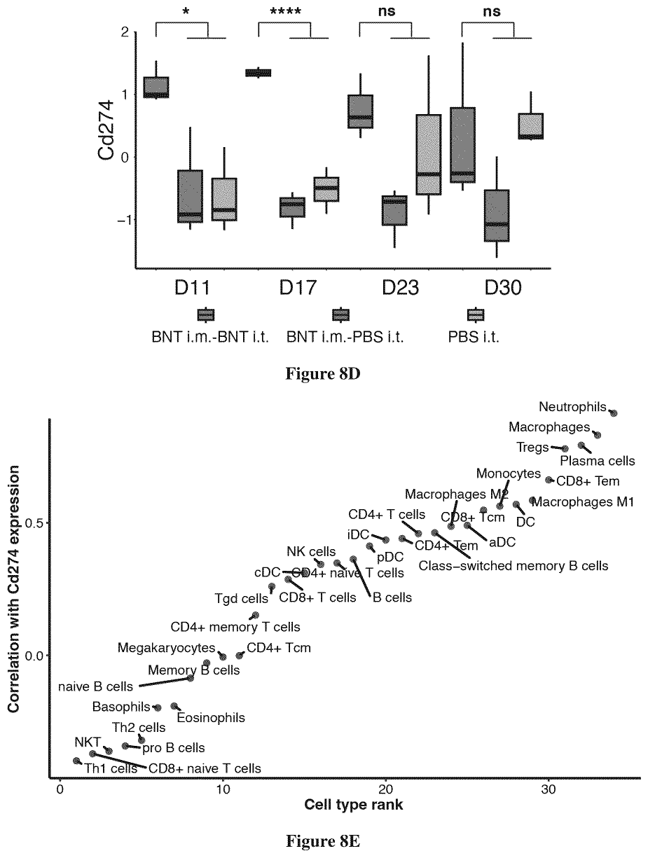

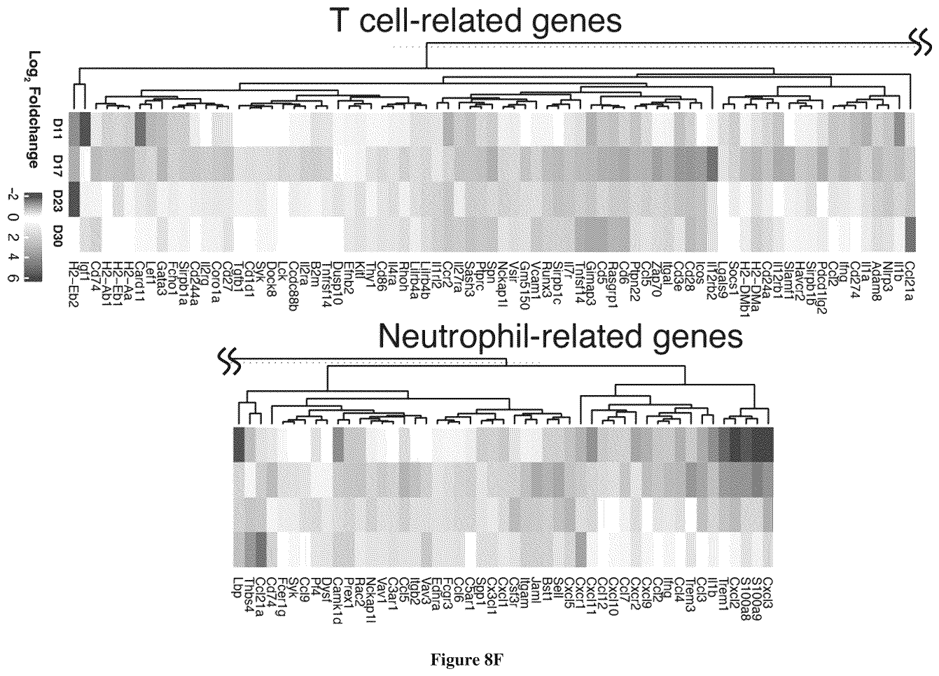

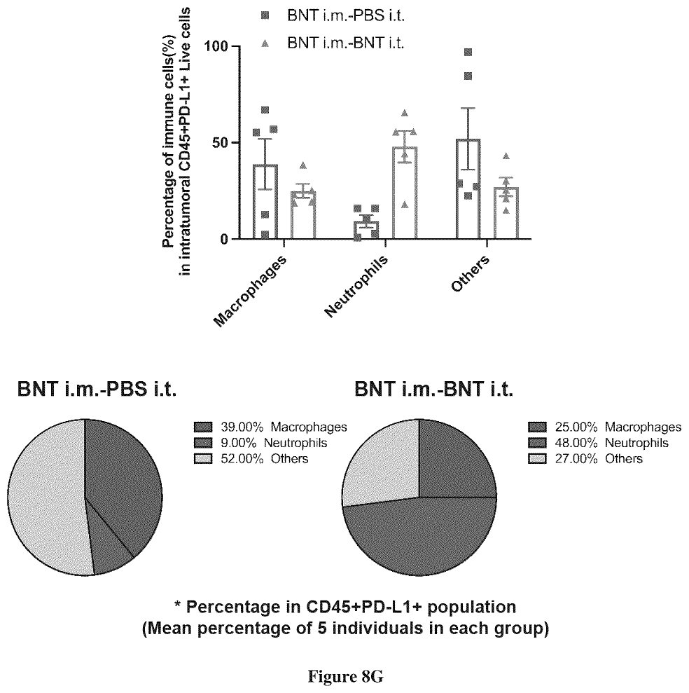

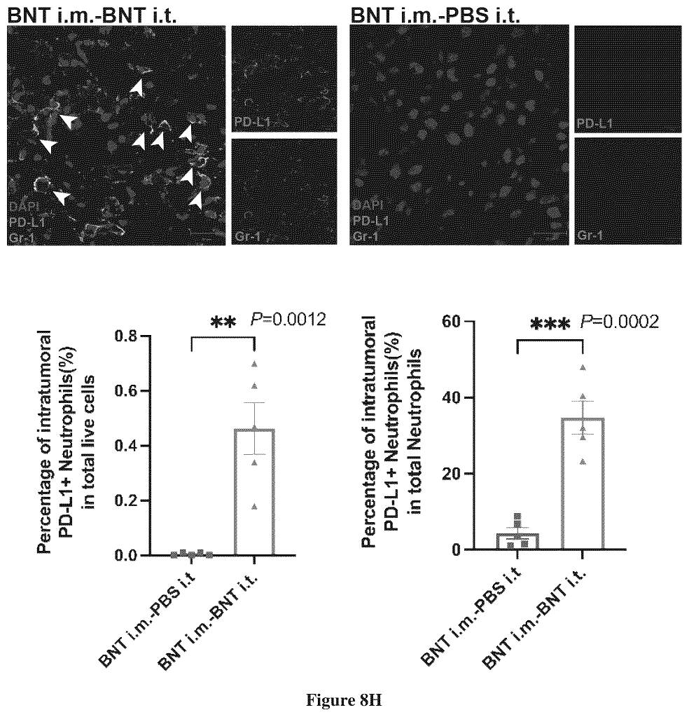

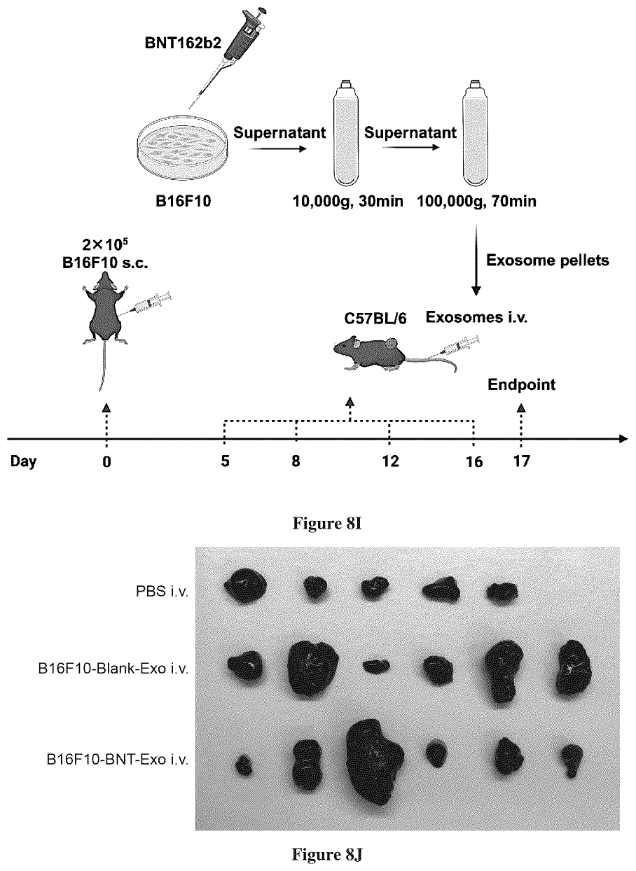

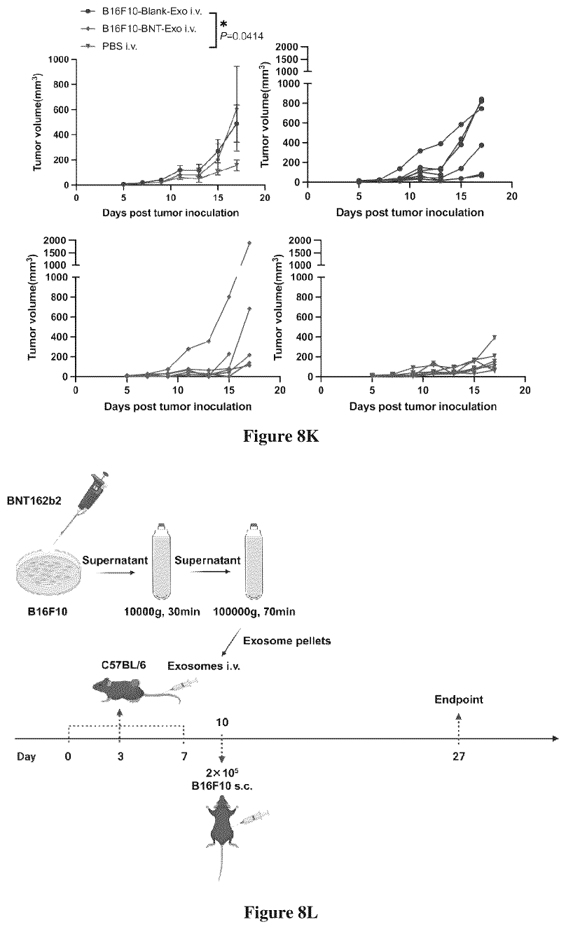

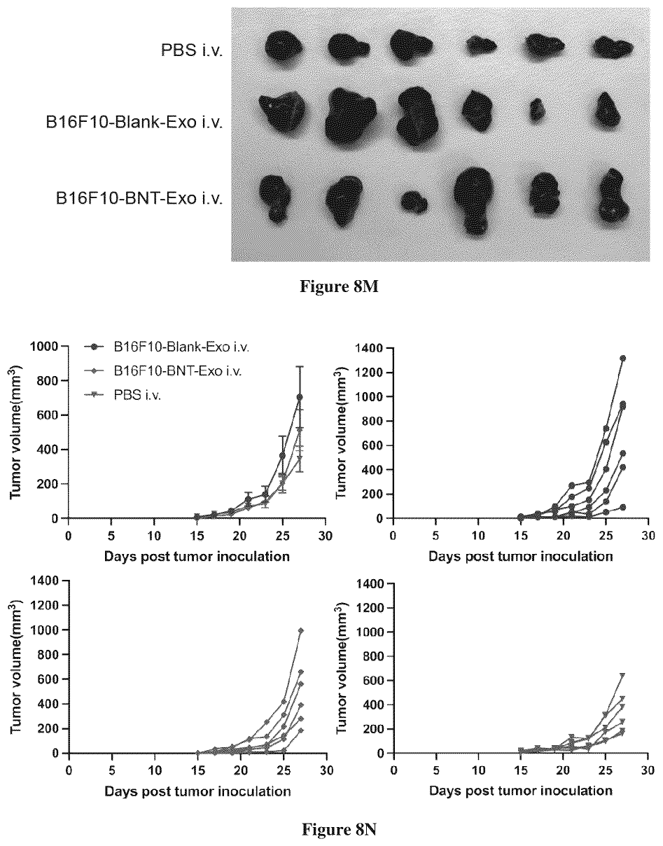

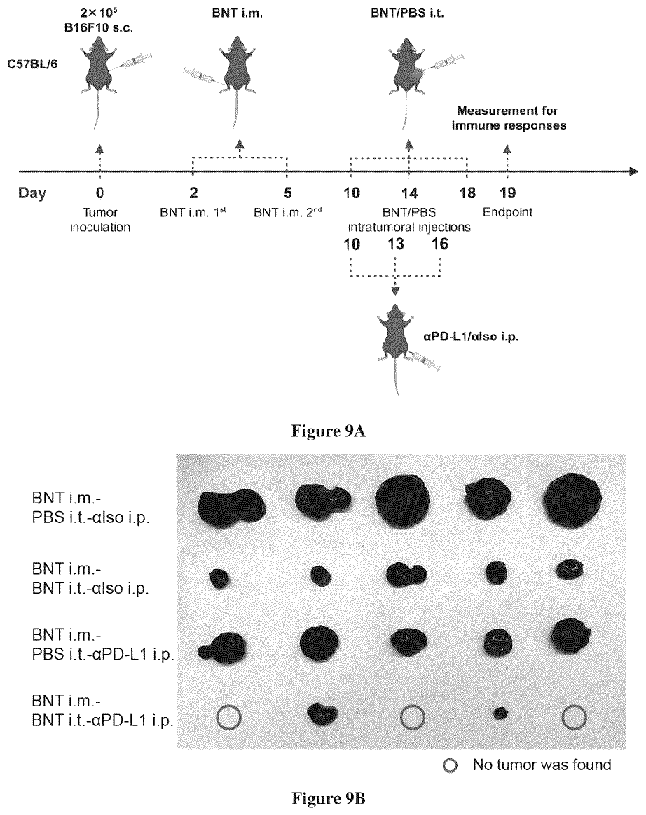

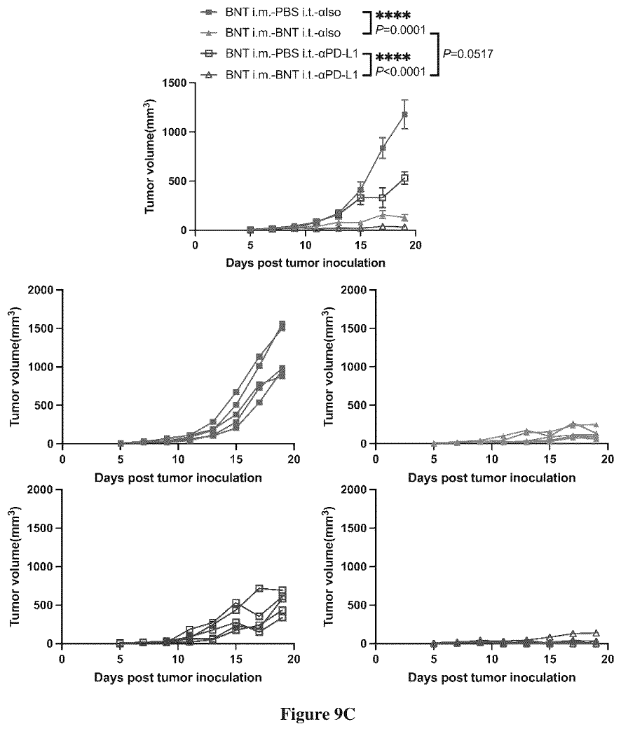

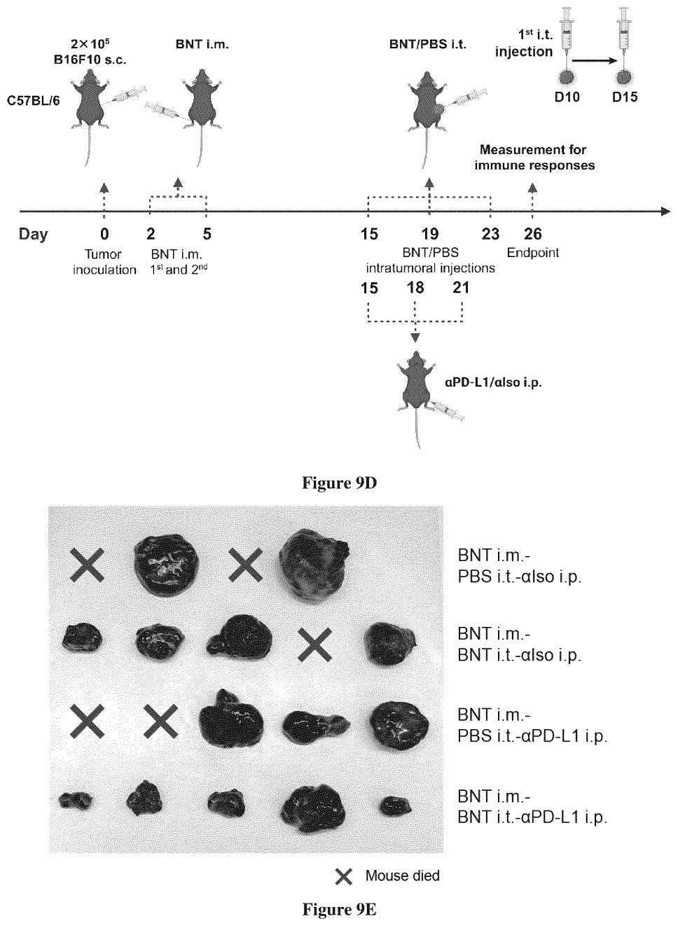

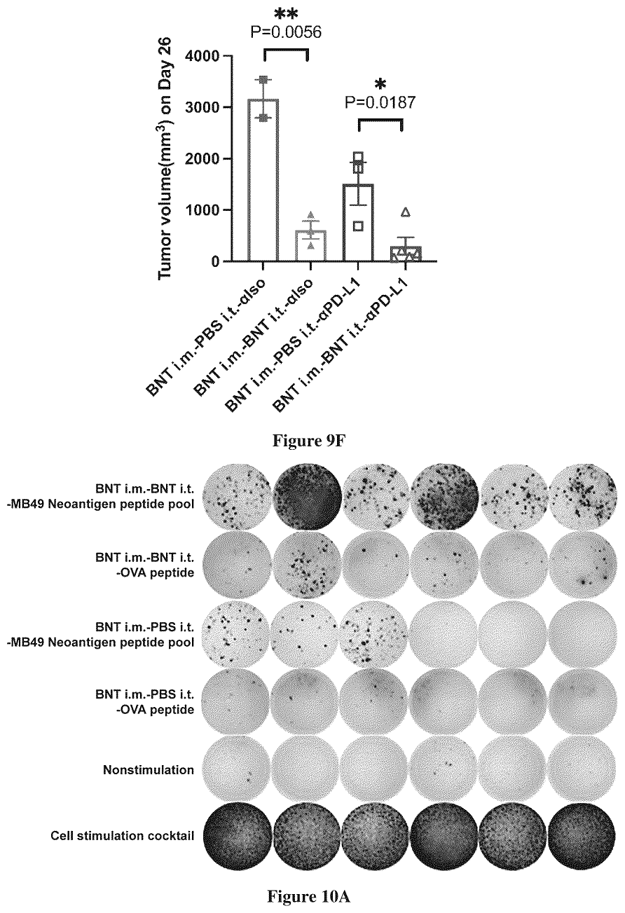

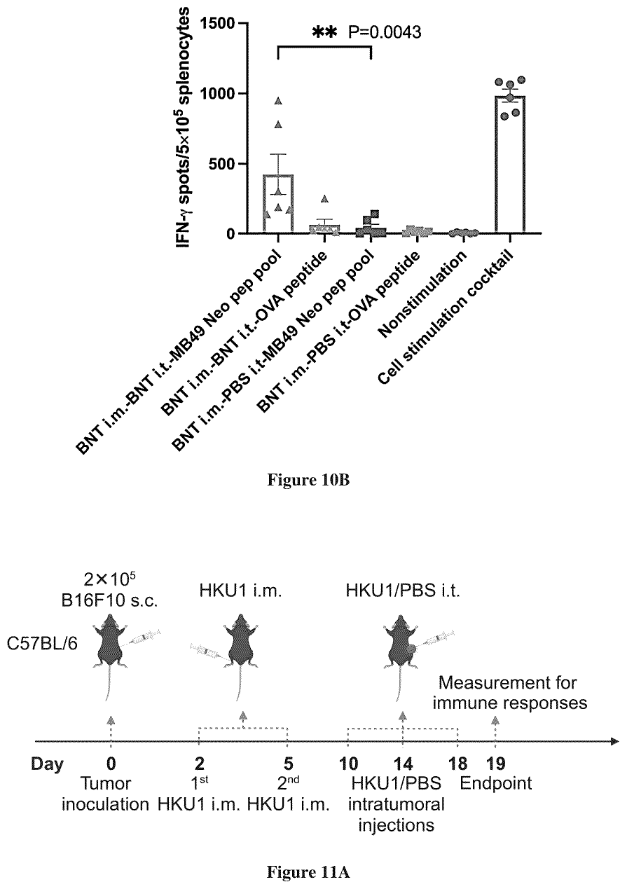

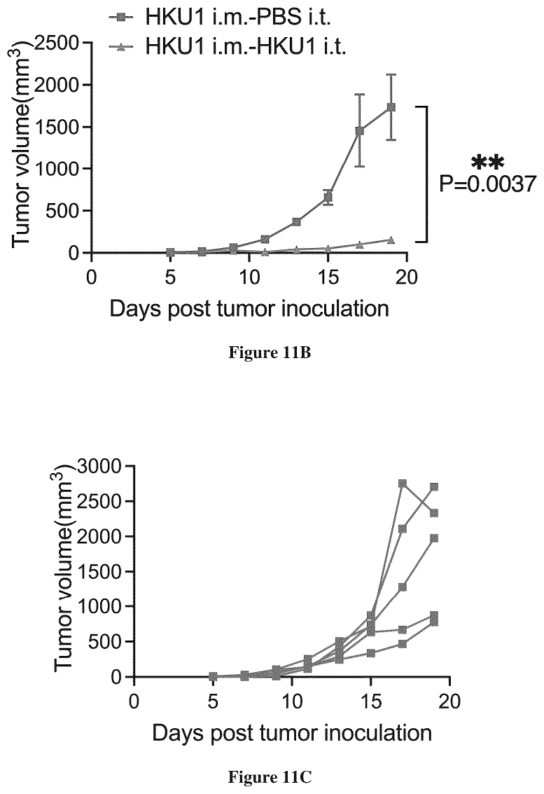

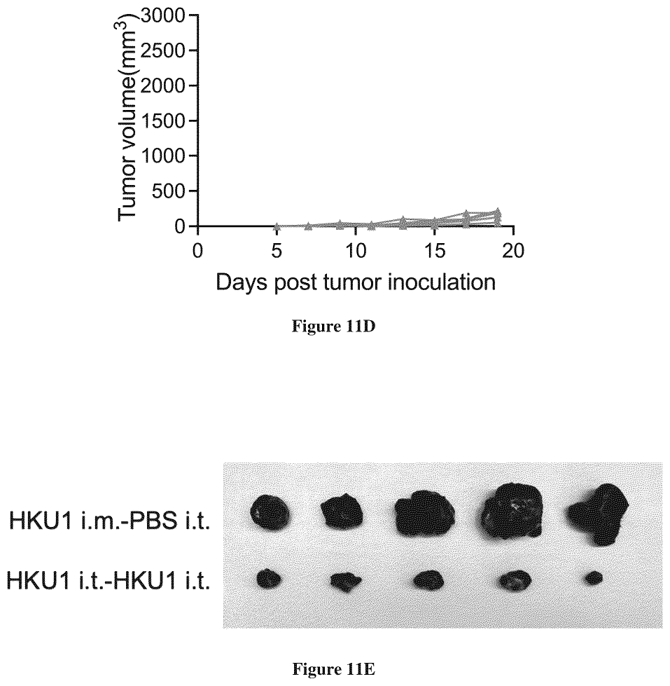

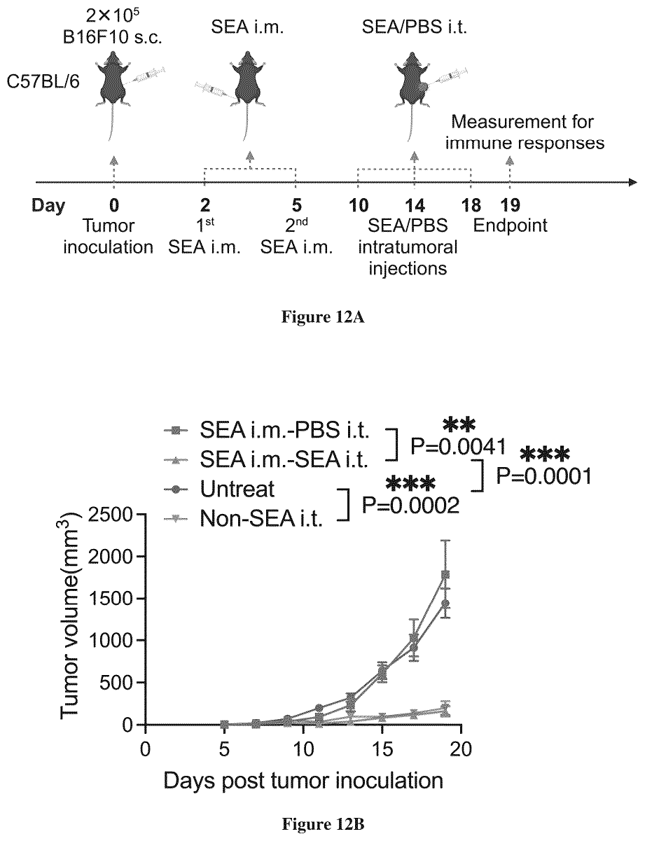

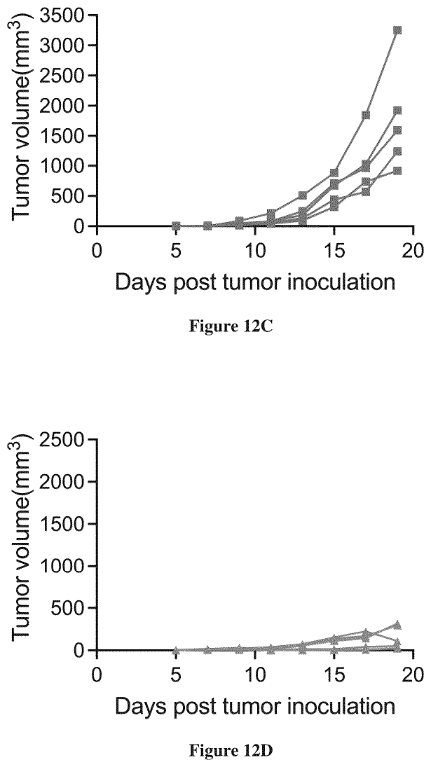

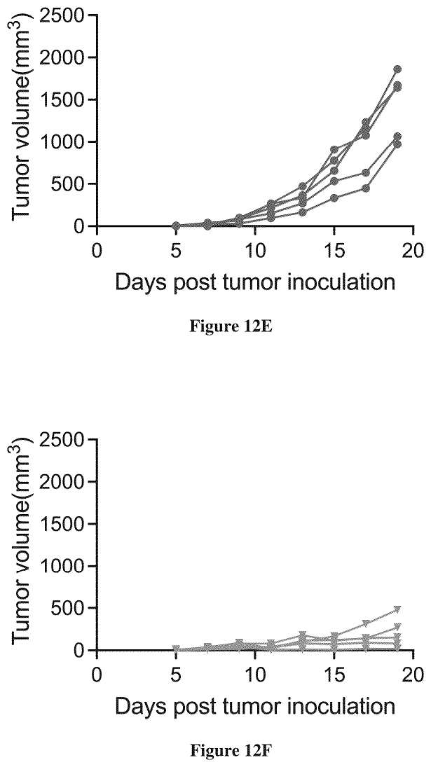

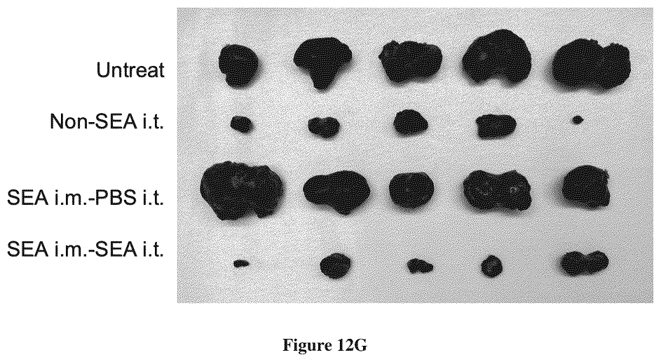

FIG. 8G shows the percentage of the PD-L1+ macrophage and the PD-L1+ neutrophil in PD-L1+ leukocytes.

FIG. 8H shows the co-localized immunofluorescent staining of PD-L1 and neutrophil marker Gr-1 in tumors from BNT16262 treated group and control group. The percentage of intratumoral PD-L1+ neutrophils in total live cells, and the percentage of intratumoral PD-L1+ neutrophils in total neutrophils.

FIG. 8I shows the experimental design of therapeutic efficacy test of BNT162b2 transfected tumor cells-derived exosomes.

FIG. 8J shows Tumor photos of therapeutic efficacy of BNT162b2 transfected tumor cells-derived exosomes.

FIG. 8K shows the tumor growth curve of therapeutic efficacy of BNT162b2 transfected tumor cells-derived exosomes in the experiment described in I.

FIG. 8L shows the experimental design of prophylactic efficacy test of BNT162b2 transfected tumor cells-derived exosomes.

FIG. 8M shows tumor photos of prophylactic efficacy of BNT16262 transfected tumor cells-derived exosomes.

FIG. 8N shows the tumor growth curve of prophylactic efficacy of BNT162b2 transfected tumor cells-derived exosomes in the experiment described in L.

FIG. 9A to 9F shows the combinational therapy of BNT162b2 and anti-PD-L1 therapy, and the therapeutic efficacy of BNT162b2 in advanced cancer.

FIG. 9A shows the experimental design of combinational therapy of BNT162b2 and anti-PD-L1.

FIG. 9B shows tumor photos of therapeutic efficacy of combinational therapy of BNT162b2 and anti-PD-L1.

FIG. 9C shows the tumor growth curve of therapeutic efficacy of combinational therapy of BNT162b2 and anti-PD-L1 in the experiment described in FIG. 9A.

FIG. 9D shows the experimental design of therapeutic efficacy of BNT162b2 in the advanced tumor model.

FIG. 9E shows the tumor photos of therapeutic efficacy of BNT162b2 and combinational therapy in the advanced tumor model.

FIG. 9F shows the tumor volume of mice in different groups at the endpoint. Experimental designs described in FIG. 9D.

FIG. 10A shows IFN-γ ELISpot showing tumor-antigen-specific T cell responses post BNT intratumoral injection therapy in MB49 tumor model.

FIG. 10B shows the bar graph showing the summary of ELISpot data in MB49 tumor model, comparing the tumor-antigen specific T cell responses in BNT162b2 intratumoral injection treatment group and PBS intratumoral injection control group. MB49 tumor neoantigen peptide pool was used as the tumor-antigen stimulator. BNT i.m.-BNT i.t: splenocytes from mice with intramuscular and intratumoral BNT162b2 injection. BNT i.m.-PBS i.t: splenocytes from mice with BNT162b2 intramuscular vaccination and intratumoral PBS injection.

FIG. 11A shows the experimental design of therapeutic efficacy of HKU1 CoV-Spike protein encoded mRNA vaccine in melanoma subcutaneous model.

FIG. 11B shows tumor growth curves of the HKU1 CoV-Spike protein encoded mRNA vaccine intratumoral injection treatment group vs. the PBS intratumoral injection control group for the experiment described in FIG. 11A.

FIG. 11C shows tumor growth curves of PBS intratumoral injection control group for the experiment described in FIG. 11A.

FIG. 11D shows tumor growth curves of the HKU1 CoV-Spike protein encoded mRNA vaccine intratumoral injection treatment group for the experiment scheme described in FIG. 11A.

FIG. 11E shows tumor comparison photo of treatment and control group from the experiment described in FIG. 11A.

FIG. 12A shows the experimental design of therapeutic efficacy of SEA protein encoded mRNA vaccine in melanoma subcutaneous model.

FIG. 12B shows tumor growth curves of the SEA protein encoded mRNA vaccine intratumoral injection post the SEA protein encoded mRNA vaccine intramuscular injection treatment group vs. the PBS intratumoral injection post the SEA protein encoded mRNA vaccine intramuscular injection control group vs. untreatmental group vs. SEA intratumoral injection only group for the experiment described in FIG. 12A.

FIG. 12C shows tumor growth curves of PBS intratumoral injection post the SEA protein encoded mRNA vaccine intramuscular injection control group for the experiment described in FIG. 12A.

FIG. 12D shows tumor growth curves of the SEA protein encoded mRNA vaccine intratumoral injection post the SEA protein encoded mRNA vaccine intramuscular injection treatment group for the experiment scheme described in FIG. 12A.

FIG. 12E shows untreatmental control group for the experiment described in FIG. 12A.

FIG. 12F shows tumor growth curves of the SEA protein encoded mRNA vaccine intratumoral injection only treatment group for the experiment scheme described in FIG. 12A.

FIG. 12G shows tumor comparison photo of treatment and control groups from the experiment described in FIG. 12A.

### DETAILED DESCRIPTION OF THE INVENTION

Vaccine-based immunotherapy against cancer faces several obstacles: weak tumor antigens, immunosuppressive tumor microenvironment, and inefficient recruitment of immune cells. The immune escape of cancer cells after anti-tumor immunotherapy further decreases the therapeutic efficacy. To overcome these obstacles, this invention provides a strategy for cancer therapy by increasing the antigenicity or immunogenicity of a tumor in a subject based on the subject's immune response due to vaccines. As an example, COVID-19 mRNA vaccines were repurposed for cancer therapy of this invention although a skilled person would readily understand that other antigens from pathogens also hold similar potential for cancer therapy as BNT162b2 in this study. Intratumoral injection of the COVID-19 mRNA vaccine re-activates the anti-spike memory immune responses and directs the anti-spike immune responses to the tumor with spike protein expression. Importantly, COVID-19 mRNA vaccine intratumoral vaccination potently inhibits the growth of the cancers tested and extends the life span of tumor-bearing mice. Careful analysis shows that COVID-19 mRNA vaccines can induce strong T cell responses against tumor-specific antigens other than the spike protein, recruit immune cells into the tumor and modify the tumor immune microenvironment. Due to the SARS-COV-2 pandemic, a large portion of the human population has been vaccinated against COVID-19. This COVID-19-vaccine cancer therapy provides a large application potential for cancer therapy in various cancer types and could be quickly translated for clinical use. In addition, the versatility of this cancer immunotherapeutic strategy was validated by using HKU1 CoV-Spike protein encoded mRNA vaccine and SEA protein encoded mRNA vaccine. The results demonstrate that the cancer immunotherapeutic strategy of this invention could be effectively applied with different mRNAs suitable for increasing antigenicity or immunogenicity of a tumor.

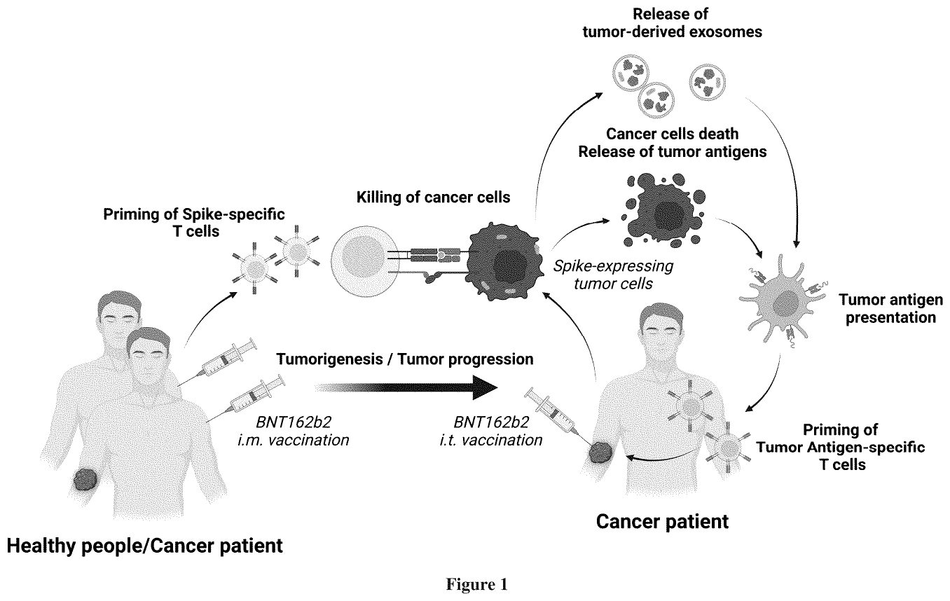

In this invention, a new cancer therapy is developed. This method utilizes the BNT162b2 vaccine not only to induce potent anti-tumor immune responses but also to convert the tumor-suppressive microenvironment from “cold” to “hot”. To achieve this goal, it was hypothesized that when the spike protein or other antigen proteins from pathogens are expressed as a tumor neoantigen in the tumor by BNT162b2 or other antigen proteins encoded mRNA vaccine intratumoral injection, the tumor can be more easily recognized by the immune system in the BNT162b2 or other antigen proteins encoded mRNA vaccine intramuscular vaccinated individuals, on the other hand, the expression of spike protein or other antigen proteins in the tumor will lead to the rapid activation of anti-spike or anti-phathogen antigens memory immune responses. At the same time, the BNT162b2 or other antigen proteins encoded mRNA vaccine intratumoral injection also helps to convert the suppressive tumor microenvironment (FIG. 1).

This invention provides a method to increase antigenicity or immunogenicity of a tumor in a subject. In one embodiment, said method comprises the step of delivering lipid nanoparticles loaded with mRNA sequences encoding one or more pathogen antigen proteins into said tumor.

In one embodiment, said subject is a human being, an animal or any living organism with a need to increase antigenicity or immunogenicity of one or more tumors.

In one embodiment, said one or more pathogen antigen proteins is an antigen targeted in a vaccine previously administered to or to be administered to said subject.

In one embodiment, said one or more pathogen antigen proteins is selected from the group consisting of coronavirus proteins, human papillomavirus proteins, respiratory syncytial virus, human immunodeficiency virus proteins, hepatitis virus, and influenza virus proteins.

In one embodiment, said lipid nanoparticle vector is delivered by intratumoral injection.

In one embodiment, said intratumoral injection is a mRNA vaccine.

In one embodiment, said mRNA vaccine is selected from the group consisting of BioNTech BNT162b2 (Comirnaty), BioNTech vaccine COVID-19 Omicron-modified Bivalent vaccine (Comirnaty Original/Omicron BA.4-5), and Moderna vaccine: mRNA-1273 (Spikevax).

In one embodiment, said mRNA vaccine is an mRNA vaccine directed against one or more antigen selected from the group consisting of bacterial antigen, bacterial superantigen, viral antigen and viral superantigen.

In one embodiment, said bacterial antigen is selected from the group consisting of CT529, CT511, CT461 (C. trachomatis), VirB9-1, VirB9-2, VirB10, conjugal transfer protein (CTP) (A. marginale), Erum0660, Erum2330, Erum2540, Erum2580, Erum5000 (E. ruminantum), OMP-19 (E. muris, E. chaffeensis), OmpA, OmpB, Adr2, YbgF. RP403, RP598 RP739, RP778, 17 T4SS-related protein C. burnetii), CBU_1835/protoporphyrinogen oxidase, CBU_1513/protoporphyrinogen oxidase, CBU_1398/SucB, CBU_0718, and CBU_0307/outer membrane protein.

In one embodiment, said viral antigen comprises one or more selected from the group consisting of: a) influenza virus hemagglutinin (HA) antigens, neuraminidase (NA) antigens, Nucleoprotein (NP), non-structural protein 1 (NSP1), non-structural protein 2/nuclear export protein (NS2/NEP), polymerase basic protein 1/2/1-F2 (PB1/2/1-F2), matrix protein 1 or matrix protein 2 of influenza virus; b) hepatitis A virus antigens (HAV) or hepatitis B virus (HBV) antigens; c) Human papillomavirus (HPV) antigens comprising major capsid (L1) protein of HPV types 6, 11, 16, 18, 31, 33, 45, 52, or 58; d) Rabies vaccine antigen comprising nucleoprotein (N), phosphoprotein (P), matrix protein (M), glycoprotein (G), or RNA-dependent RNA polymerase; e) Herpes zoster (shingles) vaccine antigen comprising varicella zoster virus glycoprotein E; f) Smallpox vaccine antigen comprising B5 antigenic domain (pB5); g) Varicella vaccine antigen comprising varicella zoster virus glycoprotein E antigen; and h) vaccine antigen for one or more of yellow fever, monkeypox, polio, mumps, Rubella or measles; i) Respiratory Syncytial Virus antigen comprising seven structural proteins (G, F. M1, M2, P, L, N) and three nonstructural proteins (NS1, NS2, SH).

In one embodiment, said bacterial superantigen or viral superantigen is selected from the group consisting of Toxic shock syndrome toxin-1 (TSST-1), Streptococcal pyrogenic exotoxin (Spe), Staphylococcal enterotoxins (SE), ETEC enterotoxin, Streptococcal superantigen (SSA), streptococcal mitogenic exotoxin (SMEZ) 1 and 2, and human endogenous retrovirus HERV-K18.1, HERV-W, MSRV.

In one embodiment, said mRNA vaccine is an mRNA vaccine directed against one or more antigens selected from the group consisting of coronavirus proteins, human papillomavirus proteins, respiratory syncytial virus, human immunodeficiency virus proteins, hepatitis virus, and influenza virus proteins.

In one embodiment, said coronavirus proteins comprises one or more of spike protein, envelope protein, membrane protein, nucleocapsid protein, accessory protein or non-structural protein of SARS-COV-1, SARS-COV-2, common cold coronaviruses, MERS or Coronavirus HuPn-2018.

In one embodiment, said common cold coronaviruses comprises one or more of HCoV-OC43, HcoV-HKU1, HcoV-229E, or HcoV-NL63.

In one embodiment, said HIV proteins comprises one or more of envelope protein, structural protein, protease, integrase, reverse transcriptase, viral protein u, viral infectivity factor, viral protein r, P6, negative regulatory factor, regulator of virion, or trans-activator of transcription of HIV (Human Immunodeficiency Virus).

In one embodiment, said influenza virus proteins comprises one or more of hemagglutinin (HA), Neuraminidase (NP), Nucleoprotein (NP), non-structural protein 1 (NSP1), non-structural protein 2/nuclear export protein (NS2/NEP), polymerase basic protein 1/2/1-F2 (PB1/2/1-F2), matrix protein 1 or matrix protein 2 of influenza virus.

In one embodiment, said method further comprises the step of inoculating said subject against said one or more pathogen antigen proteins or their variants.

In one embodiment, said subject has been previously inoculated against said one or more pathogen antigen proteins or their variants.

In one embodiment, said tumor is selected from the group consisting of melanoma, breast cancer, bladder cancer, colon cancer, gastric cancer, pancreatic cancer, blood cancer, lung cancer, and liver cancer.

This invention also provides a method to treat a subject having one or more tumors. In one embodiment, said method comprises increasing antigenicity or immunogenicity of at least one tumor among said one or more tumors using the method of this invention.

In one embodiment, said at least one tumor is a primary tumor or secondary tumor.

In one embodiment, said one or more tumors comprises primary tumor and secondary tumor.

In one embodiment, said method further comprises co-administering an immune checkpoint inhibitor to said subject.

In one embodiment, said immune checkpoint inhibitor is administered intratumoral, intraperitoneally or intravenously. In another embodiment, said immune checkpoint inhibitor is administered by any administration route suitable for said immune checkpoint inhibitor.

In one embodiment, said immune checkpoint inhibitor is one or more selected from the group consisting of anti-PD-1, anti-CTLA4, anti-PD-L1.

In one embodiment, said subject is an advanced cancer patient. In another embodiment, this invention also indicated the therapeutic efficacy in the advanced cancer.

This invention further provides an intratumoral injection for treating tumor in a subject. In one embodiment, said intratumoral injection comprises lipid nanoparticles loaded with mRNA encoding one or more pathogen antigen proteins.

In one embodiment, said one or more pathogen antigen proteins is an antigen targeted in a vaccine previously administered to or to be administered to said subject.

In one embodiment, said tumor is selected from the group consisting of melanoma, breast cancer, bladder cancer, colon cancer, gastric cancer, pancreatic cancer, blood cancer, lung cancer, and liver cancer.

In one embodiment, said intratumoral injection is an mRNA vaccine directed against one or more antigens selected from the group consisting of bacterial antigen, bacterial superantigen, viral antigen and viral superantigen.

In one embodiment, said one or more pathogen antigen proteins is selected from the group consisting of bacterial antigen, bacterial superantigen, viral antigen and viral superantigen.

In one embodiment, said bacterial antigen is selected from the group consisting of CT529, CT511, CT461 (C. trachomatis), VirB9-1, VirB9-2, VirB10, conjugal transfer protein (CTP) (A. marginale), Erum0660, Erum2330, Erum2540, Erum2580, Erum5000 (E. ruminantum), OMP-19 (E. muris, E. chaffeensis), OmpA, OmpB, Adr2, YbgF, RP403, RP598 RP739, RP778,17 T4SS-related proteins (C. burnetii), CBU_1835/protoporphyrinogen oxidase, CBU_1513/protoporphyrinogen oxidase, CBU_1398/SucB, CBU_0718, and CBU_0307/outer membrane protein.

In one embodiment, said viral antigen comprises one or more selected from the group consisting of: a) influenza virus hemagglutinin (HA) antigens or neuraminidase (NA) antigens; b) hepatitis A virus antigens (HAV) or hepatitis B virus (HBV) antigens; c) Human papillomavirus (HPV) antigens comprising major capsid (L1) protein of HPV types 6, 11, 16, 18, 31, 33, 45, 52, or 58; d) Rabies vaccine antigen comprising nucleoprotein (N), phosphoprotein (P), matrix protein (M), glycoprotein (G), or RNA-dependent RNA polymerase; e) Herpes zoster (shingles) vaccine antigen comprising varicella zoster virus glycoprotein E; f) Smallpox vaccine antigen comprising B5 antigenic domain (pB5); g) Varicella vaccine antigen comprising varicella zoster virus glycoprotein E antigen; and h) vaccine antigen for one or more of yellow fever, polio, mumps, or measles.

In one embodiment, said bacterial superantigen or viral superantigen is selected from the group consisting of Toxic shock syndrome toxin-1 (TSST-1), Streptococcal pyrogenic exotoxin (Spe), Staphylococcal enterotoxins (SE), ETEC enterotoxin, Streptococcal superantigen (SSA), streptococcal mitogenic exotoxin (SMEZ) 1 and 2, and human endogenous retrovirus HERV-K18.1, HERV-W, MSRV.

In one embodiment, said one or more pathogen antigen proteins is selected from the group consisting of coronavirus proteins, HIV proteins and influenza virus proteins.

In one embodiment, said coronavirus proteins comprises one or more of spike protein, envelope protein, membrane protein, nucleocapsid protein, accessory protein or non-structural protein of SARS-COV-1, SARS-COV-2, common cold coronaviruses, MERS or Coronavirus HuPn-2018.

In one embodiment, said common cold coronaviruses comprises one or more of HCoV-OC43, HCoV-HKU1, HCOV-229E, or HCoV-NL63.

In one embodiment, said HIV proteins comprises one or more of envelope protein, structural protein, protease, integrase, reverse transcriptase, viral protein u, viral infectivity factor, viral protein r, P6, negative regulatory factor, regulator of virion, or trans-activator of transcription of HIV (Human Immunodeficiency Virus).

In one embodiment, said influenza virus proteins comprises one or more of hemagglutinin (HA), Neuraminidase (NP), Nucleoprotein (NP), non-structural protein 1 (NSP1), non-structural protein 2/nuclear export protein (NS2/NEP), polymerase basic protein 1/2/1-F2 (PB1/2/1-F2), matrix protein 1 or matrix protein 2 of influenza virus.

In one embodiment, said one or more pathogen antigen proteins is an antigen targeted in a vaccine.

In one embodiment, said vaccine is selected from the group consisting of Bacilli Calmette-Guérin vaccine (attenuated live bovine tuberculosis bacillus, Mycobacterium bovis), Diphtheria, Tetanus, and Pertussis vaccine (DTaP) (Tetanus Toxoid, Reduced Diphtheria Toxoid and Adsorbed Acellular Pertussis), MenACWY vaccine (The MenACWY vaccine offers protection against 4 types of bacteria that can cause meningitis: meningococcal groups A, C, W and Y.), MenB vaccine, Hib/MenC vaccine (Haemophilus influenzae type b (Hib) and meningitis C), Smallpox vaccine (variola virus (often called smallpox virus)), Monkeypox vaccine (mpox virus), Polio vaccine, Inactivated poliovirus vaccine, Human hepatitis A vaccine, Human hepatitis B, Measles-Mumps-Rubella vaccine (MMR), and Varicella vaccine (Varicella (chickenpox)).

In one embodiment, said antigen targeted in Bacilli Calmette-Guerin vaccine comprises one or more of cell surface glycolipoprotein MPB63, MPB70, MPB83, Outer membrane channel protein CpnT, and other antigens.

In one embodiment, said antigen targeted in Diphtheria, Tetanus, and Pertussis vaccine (DTaP) comprises one or more of Diphtheria toxin (consisting of two subunits linked by disulfide bridges, known as A and B toxin); Tetanus Toxoid (consisting of one light chain (50-kDa) and one heavy chain (100-kDa)); Pertussis toxin (it consists of five different subunits, designated S1, S2, S3, two S4 hexamer, and S5).

In one embodiment, said antigen targeted in MenACWY vaccine comprises one or more of antigens in meningococcal groups A, C, W and Y.

In one embodiment, said antigen targeted in MenB vaccine comprises one or more of antigens in meningococcal group B bacteria.

In one embodiment, said antigen targeted in Hib/MenC vaccine comprises one or more of antigens in Haemophilus influenzae type b (Hib) and meningitis C.

In one embodiment, said antigen targeted in smallpox vaccine comprises one or more of Virion membrane protein OPG144 precursor/OPG141/OPG135/OPG105/OPG143/OPG140, Envelope protein H3/OPG155, Envelope phospholipase OPG057, Superinfection exclusion protein (OPG040), Chemokine-binding protein (OPG001 (B29R, C23L)), Cell surface-binding protein OPG105, Major core protein OPG136 precursor/OPG130/VP8/D2, Scaffold protein D13, and all proteins in Smallpox.

In one embodiment, said antigen targeted in Monkeypox vaccine comprises one or more of OPG105, OPG077, DNA-directed RNA polymerase 132 kDa polypeptide (RPO132), Profilin (OPG171), p28, OPG101 (TK), OPG188 (B4R), Cu—Zn superoxide dismutase-like protein (A46R). Virion membrane protein OPG140/OPG139/OPG135/OPG144 precursor, Core protein OPG142/OPG138/OPG130/OPG129 and other antigens in Monkeypox.

In one embodiment, said antigen targeted in Polio vaccine or Inactivated poliovirus vaccine comprises one or more of Capsid protein VP0/VP1/VP2/VP3/VP4, Protease 2A, Protein 2B, Protein 2C, Protein 3AB, Protein 3A, Protein 3CD, Protease 3C, and other antigens.

In one embodiment, said antigen targeted in Human hepatitis A vaccine comprises one or more of Capsid protein VP0/VP1/VP2/VP3/VP4, Protein VP1-2A, Protein 2B, Protein 2BC, Protein 2C, Protein3ABC, Protein 3A, Protein 3CD, Protease 3C Viral protein genome-linked, and other antigens.

In one embodiment, said antigen targeted in Human hepatitis B vaccine comprises one or more of External core antigen C, Large envelope protein S, Capsid protein C, Protein P, Protein X, and other antigens.

In one embodiment, said antigen targeted in Measles-Mumps-Rubella vaccine (MMR) comprises one or more measles antigens selected from the group consisting of Non-structural protein V. Nucleoprotein N, Hemagglutinin glycoprotein H, Fusion glycoprotein FO, RNA-directed RNA polymerase L, Matrix protein M, Phosphoprotein P/V, and other antigens; one or more mumps antigens selected from the group consisting of Fusion glycoprotein FO, Non-structural protein V, RNA-directed RNA polymerase L, Hemagglutinin-neuraminidase HN, Nucleoprotein N (NP). Small hydrophobic protein (SH), and other antigens; one or more Rubella antigens selected from the group consisting of Non-structural polyprotein p200, Structural polyprotein, and other antigens.

In one embodiment, said antigen targeted in Varicella vaccine comprises one or more of Envelope glycoprotein B, Envelope glycoprotein E. Capsid scaffolding protein 33, Envelope protein US9. Envelope glycoprotein C, Envelope glycoprotein I. Large tegument protein deneddylase, Major viral transcription factor ICP4 homolog, Envelope glycoprotein H, Triplex capsid protein 1, Small capsomere-interacting protein SCP, Envelope glycoprotein M, Envelope glycoprotein N. Major DNA-binding protein DBP, Envelope glycoprotein K, Cytoplasmic envelopment protein 1, Cytoplasmic envelopment protein 2, Cytoplasmic envelopment protein 3, Envelope glycoprotein L, Structural protein 1, Membrane protein 0, Envelope glycoprotein H, Nuclear egress protein 2, Capsid vertex component 1, Capsid vertex component 2, Packaging protein UL32, Portal protein, and other antigens.

This invention also provides a kit for treating tumor in a subject. In one embodiment, said kit comprises the intratumoral injection of this invention.

In one embodiment, said kit further comprises a vaccine for inoculating said subject against said one or more pathogen antigen proteins before or after said intratumoral injection is administered.

In one embodiment, said kit further comprises an immune checkpoint inhibitor.

### Example 1

### Methods and Materials

### 1) Cancer Cell Lines and Vaccine

Mouse 4T1 breast cancer cell line, mouse B16F10 melanoma cancer cell line and mouse MB49 bladder cancer cell line were used.

4T1 and B16F10 cell lines were cultured in RPMI Medium 1640 with 10% Fetal Bovine Serum (FBS). MB49 cell line and CT26 cell line were cultured in Dulbecco's Modified Eagle Medium (DMEM) medium with 10% Fetal Bovine Serum (FBS). All cell lines were cultured at 37° C., 5% CO2.

Leftover of Pfizer/BioNTech BNT162b2 mRNA vaccine (Comirnaty) after patient use was acquired in the original vials from the Department of Health, HKSAR. Lipid nanoparticle encapsulated HKU1 CoV-Spike protein encoded mRNA vaccine and lipid nanoparticle encapsulated SEA protein encoded mRNA vaccine were developed in-house.

### 2) Mice, Tumor Challenge, and Vaccination

3×10⁵ and 2×10⁵ B16F10 tumor cells were subcutaneously inoculated in female C57BL/6 mice for the ‘BNT162b2 vaccination before cancer inoculation’ and the ‘BNT162b2 vaccination after cancer inoculation’ models, respectively. Diluted BNT162b2 (30 ug mRNA in 1.8 ml saline) was intramuscular administrated 50 μl per mouse before or after tumor inoculation. 50 μl diluted BNT162b2 was administrated by intratumoral injection for each mouse; for control group mice, 50 μl PBS was administrated by intratumoral injection.

1×10⁵. MB49 tumor cells were subcutaneously inoculated in male C57BL/6 mice for the ‘BNT162b2 vaccination after cancer inoculation’ models. Diluted BNT16262 was intramuscular administrated 50 μl per mouse after tumor inoculation. 50 μl diluted BNT162b2 was administrated by intratumoral injection for each mouse; for control group mice, 50 μl PBS was administrated by intratumoral injection.

2×10⁵ 4T1 tumor cells were subcutaneously inoculated in female BalB/c mice for the ‘BNT162b2 vaccination after cancer inoculation’ models. Diluted BNT162b2 was intramuscular administrated 50 μl per mouse after tumor inoculation. 50 μl diluted BNT162b2 was administrated by intratumoral injection for each mouse; for control group mice, 50 μl PBS was administrated by intratumoral injection.

1.75×10⁵ CT26 tumor cells were subcutaneously inoculated in female BalB/c mice for the ‘BNT162b2 vaccination after cancer inoculation’ models. Diluted BNT162b2 was intramuscular administrated 50 μl per mouse after tumor inoculation. 50 μl diluted BNT162b2 was administrated by intratumoral injection for each mouse; for control group mice, 50 μl PBS was administrated by intratumoral injection.

### 3) Cytokine Profiling

Blood samples and tumor tissues were collected from mice in the BNT162b2 treatment group and the control group at the endpoint for cytokine profiling (BioLegend, LEGENDplex MU Th Cytokine Panel [12-plex], 741043 and LEGENDplex Mouse Inflammation Panel [13-plex], 740446).

### 4) RNA-Seq and Data Analysis

Total RNA was extracted from tumor tissues by Trizol and chloroform followed by precipitation in 2-propanol and ethanol. Gene expression was quantitated by “Salmon” directly from the bulk transcriptome sequencing data. DESeq2 was used for the differential analysis of gene expression using default parameters. Gene ontology (GO) analysis was limited to immune-related biology processes (ontologies belonging to GO:0002376) and GO terms were merged according to the similarity between each ontology with the help of clusterProfiler. Immune cell functional enrichment was conducted by xCell using a mouse gene expression profile with the mouse gene symbol transferred to the human gene symbol.

### 5) ELISA Assay

96-well ELISA plates (JET BIOFIL, FEP-100-096) were coated overnight with 0.1 ug/ml Spike protein or 10 ug/ml Tumor membrane protein in coating buffer. The plates were blocked by Blocker in 1×TBST and incubated at room temperature for 2 hours. Serum from each group was 1:150 diluted in Blocker buffer for Spike protein ELISA and 1:5 diluted in blocker buffer for tumor membrane protein ELISA, and plates were incubated at room temperature for 2 hours. After washing by 1×TBST three times, horseradish peroxidase (HRP)-conjugated goat anti-mouse IgG (1:5000, GE Healthcare) was incubated at room temperature for 1 hour. The plates were then washed by 1×TBST five times. 100 μl of HRP substrate (TMB Chromogen Solution (for ELISA), 002023) was added to each well. After 15 min incubation, adding 50 μl of 2M H2SO4 solution to stop the reaction, the ELISA plates were analyzed by an absorbance microplate reader (Varioskan Flash, Thermo Scientific) at 450 nm wavelength. Data analysis was done by GraphPad Prism to calculate ELISA AUC.

### 6) ELISpot Assay

ELISpot assay was performed using Mouse IFN-γ ELISpot PLUS (HRP) Kit (Mabtech, 3321-4HST-2). Splenocytes were obtained from sacrificed mice in BNT intratumoral injection therapy group and control group. Splenocytes were stimulated by spike protein peptide pool, B16F10 cell lysate, and ML-1 cell lysate, respectively. Splenocytes with stimulators were incubated overnight at 37° ° C. ELISpot plates were imaged by CTL ImmunoSpot ELISpot Analyzer. Data were statistically analyzed by Student's t-test.

### 7) Flow Cytometry

After obtaining the tumor tissues from sacrificed mice in each group, tumor tissues were digested by Collagenase I and IV (Sigma-Aldrich, SCR103 and C5138), digested single cells were stained by Anti-mouse CD45-Brilliant Violet 605, Anti-mouse CD3-FITC, Anti-mouse CD19-Brilliant Violet 421 (Biolegend). Flow Cytometry and data analysis were performed on an Agilent NovoCyte Quanteon analyzer.

### 8) Immunofluorescence Staining

Tumors obtained from sacrificed mice were embedded in the Tissue-Tek O.C.T Compound (SAKURA, 4583), tumor frozen section was performed by Thermo NX50. After blocking the tumor sections with 5% BSA, tumor sections were stained by SARS Coronavirus Spike Protein Polyclonal Antibody (Thermo Fisher, PA1-41165) at 4° C. overnight, and slides were washed by 1×TBST 3 times, 5 min each time, then slides were stained by Goat anti-Rabbit IgG(H+L) Secondary Antibody, Alexa Fluor 488 (Thermo Fisher, A-11008) 1 hour at room temperature, slides were washed by 1×TBST 3 times, 5 min each time. After that, DAPI was stained for 20 min at room temperature, and slides were washed by 1×TBST 3 times, 5 min each time. Images were acquired using Carl Zeiss LSM880.

### 8) Construction of OVA High Expressional Tumor Cell Line, Cell Line Transfection

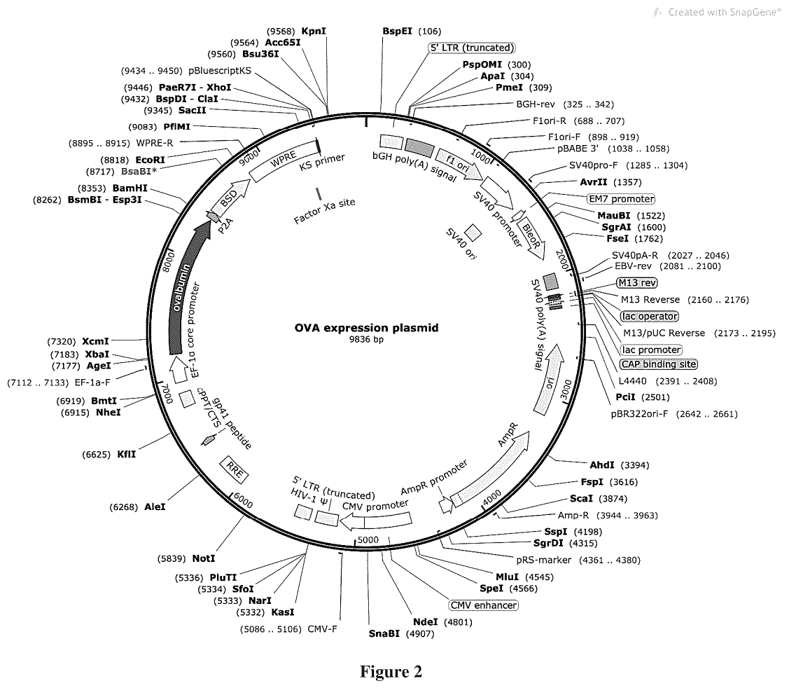

Chicken ovalbumin lentivirus was packaged by LentiX cell line using the plasmid as the map shown in FIG. 2, chicken ovalbumin lentivirus was collected twice every 24 hours, and the collected lentivirus was concentrated by ultracentrifuge, 126100×g. 2 hours, 4° C. B16F10-OVA cell line was constructed by infection of concentrated chicken ovalbumin lentivirus. The transfected B16F10 cell line with OVA high expressional level was sorted by flow cytometry (BD FACSAria Fusion).

1.2 ug BNT162b2 mRNA was added into 2-3×10⁶ B16F10-OVA directly, the medium was changed at 8 hours post-transfection, and the medium was collected 2-3 times every 24 hours, all collected medium was stored at 4° ° C., and all exosome in the medium was isolated together by ultracentrifuge.

### 9) Exosome Isolation

Cells were cultured in RPMI-1640 containing 1% penicillin-streptomycin and 10% exosome-depleted FBS (Thermo Scientific, A2720803), the medium was collected every two days, three times. Firstly, the medium was centrifuged at 2,000 g. 20 min, then the supernatant was filtered through a 0.45 μm filter, the flowthrough was centrifuged at 10,000 g. 30 min, then the cellular debris was totally removed in the supernatant. Exosome isolation was performed by ultracentrifugation at 100,000 g. 70 min, then discarding all supernatant and resuspending the pellets on the bottom of the ultracentrifuge tube using PBS, and ultracentrifuges at 100,000, 70 min again to collect the pellets in PBS, and stored in −80° C.

### 10) Western Blot

B16F10 or B16F10-OVA derived exosomes were lysed in Mammalian Protein Extraction Reagent (78501, Thermo Scientific) with protease inhibitor cocktail (04693132001, Roche). The proteins were separated by SDS-PAGE followed by transferring proteins to PVDF membranes. After 60 min blocking by 5% Blotting-Grade Blocker (BIO-RAD, 1706404), the PVDF membranes were incubated with primary antibodies overnight at 4° C. Then the secondary antibody, horseradish peroxidase-conjugated anti-rabbit or anti-mouse IgG (ThermoFisher, 31460, 31430) was incubated with membranes for 1 h at room temperature. The protein bands were finally detected by enhanced chemiluminescence (ECL) detection system (ThermoFisher, Pierce ECL Western Blotting Substrate, 32106).

### Example 2

### Results

### 1) BNT162b2 has a Potent Cancer Therapy Efficacy.

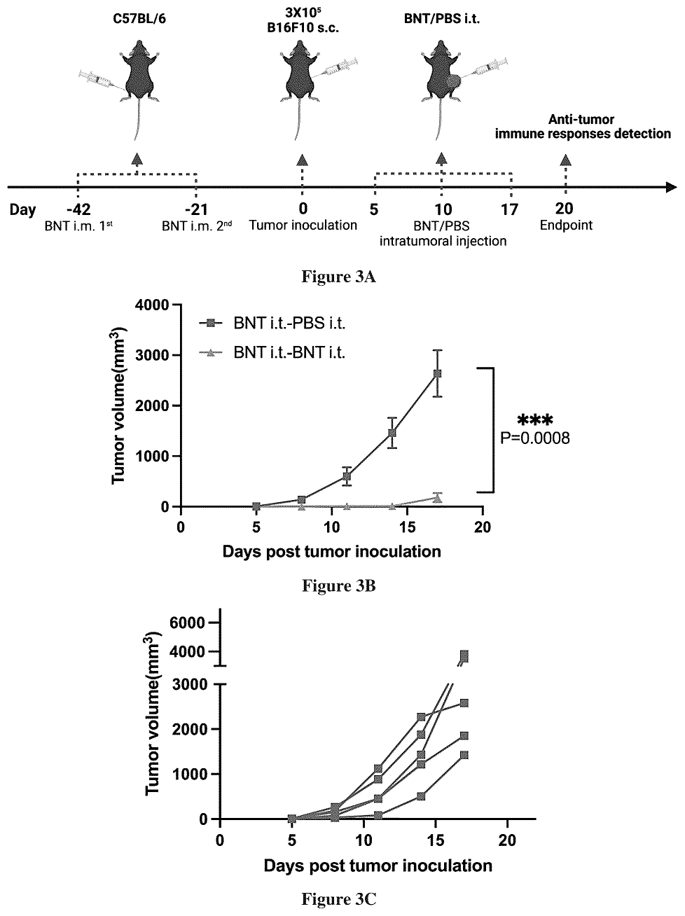

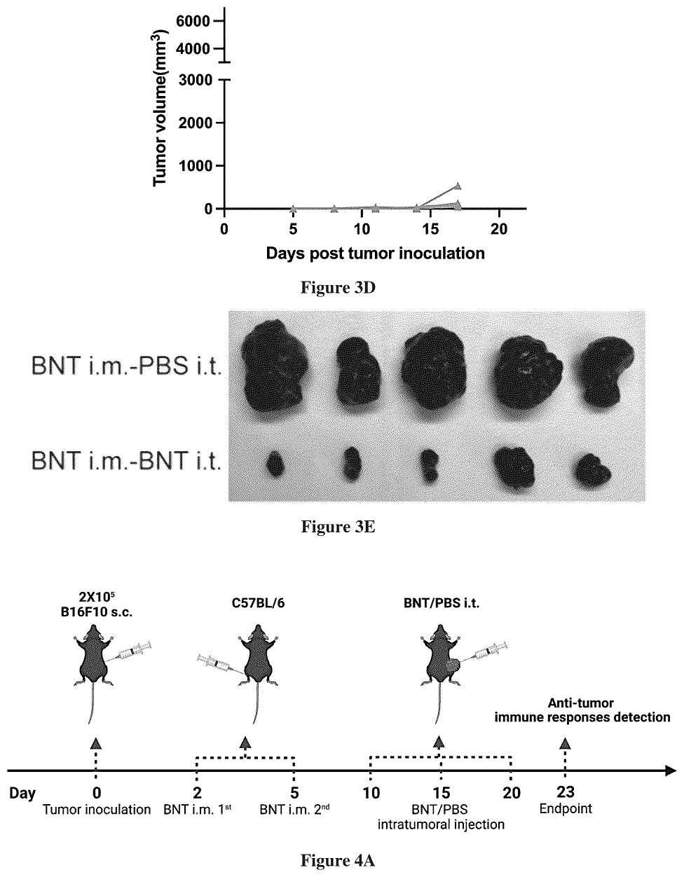

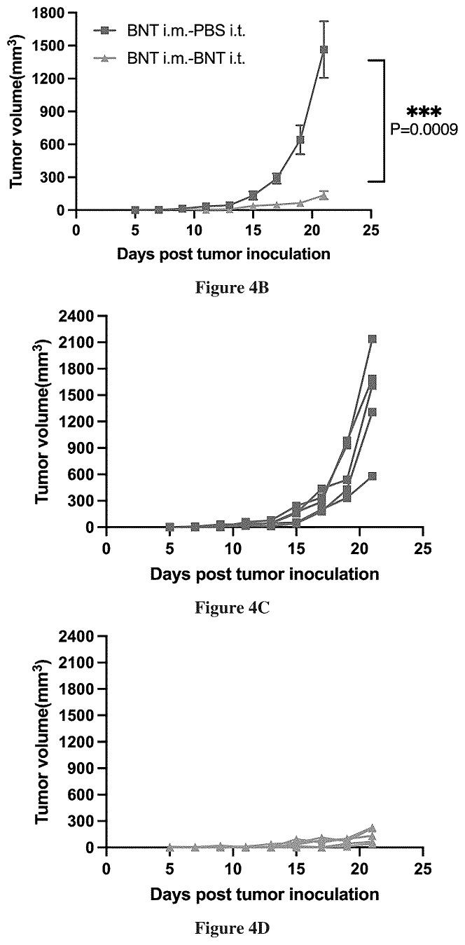

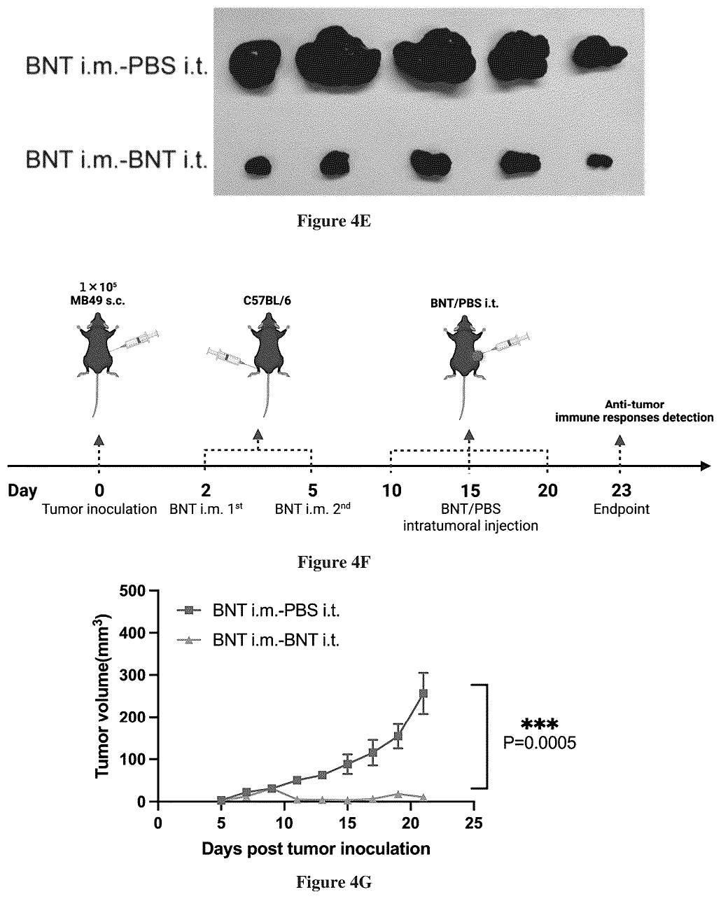

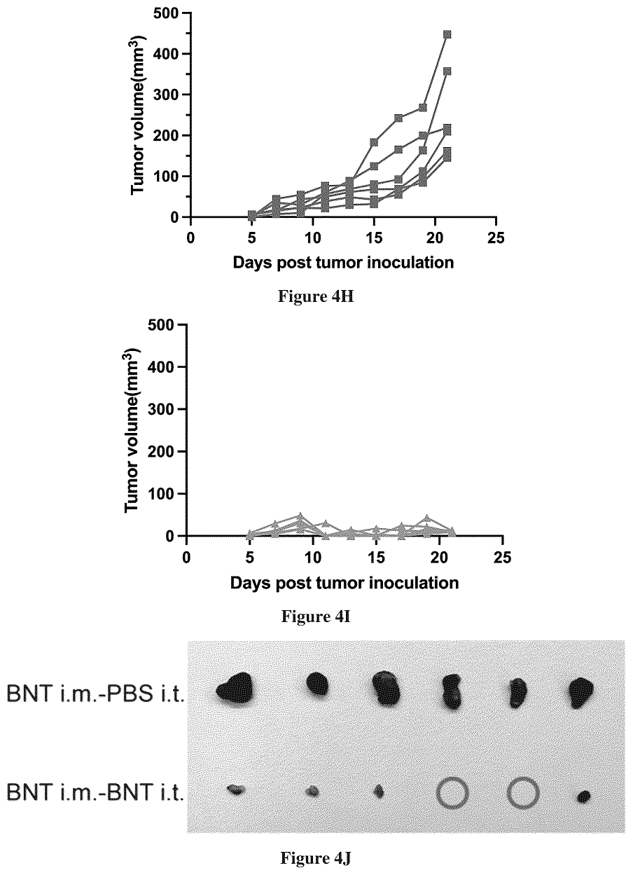

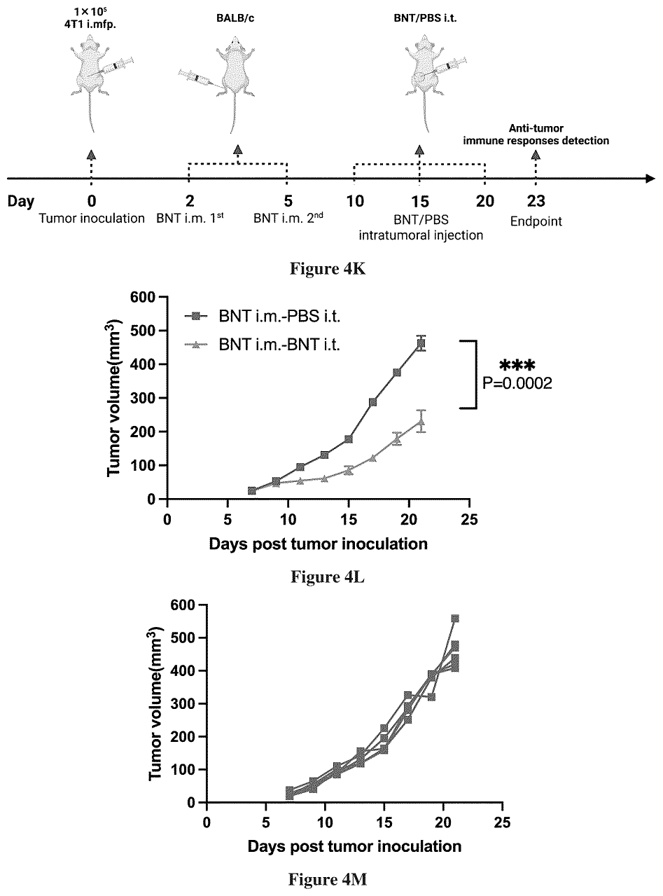

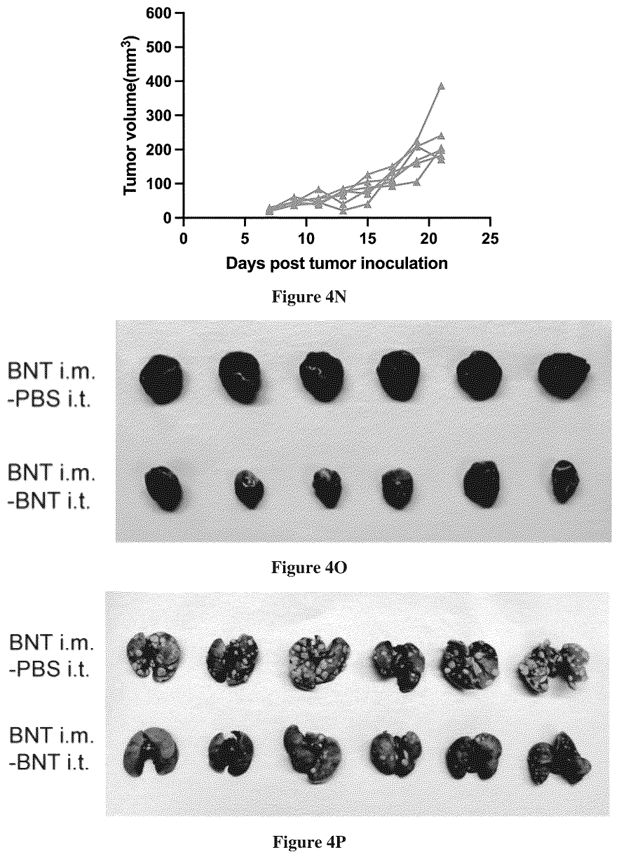

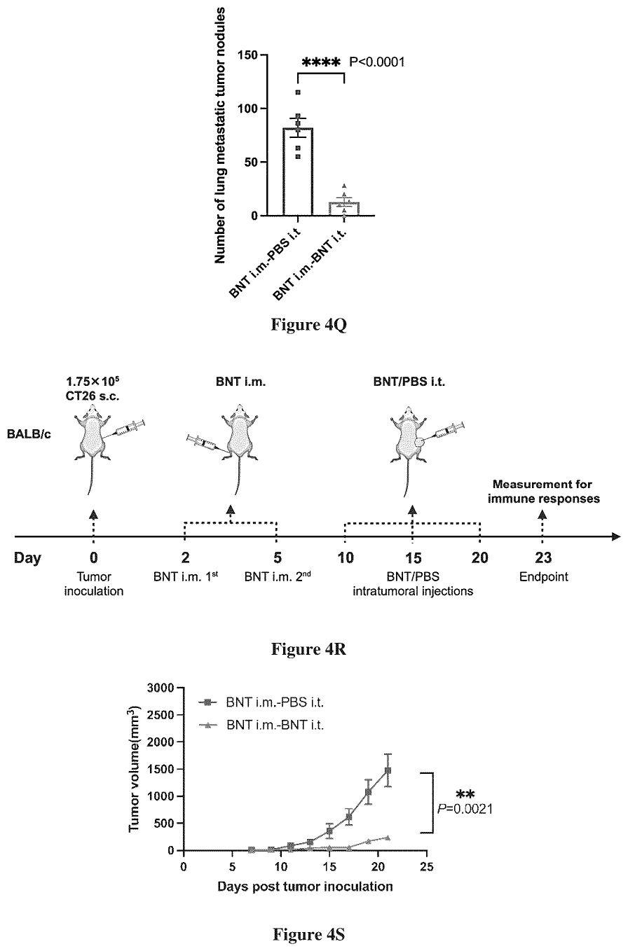

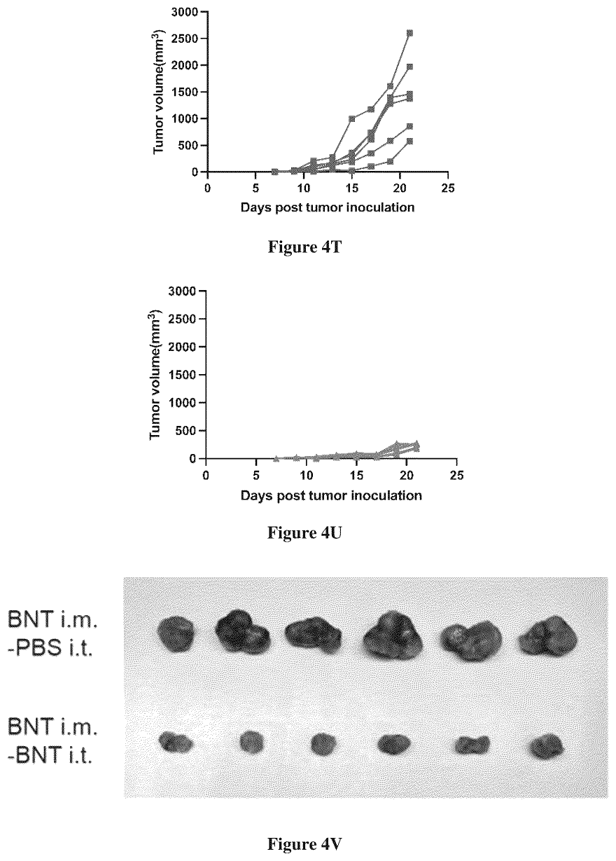

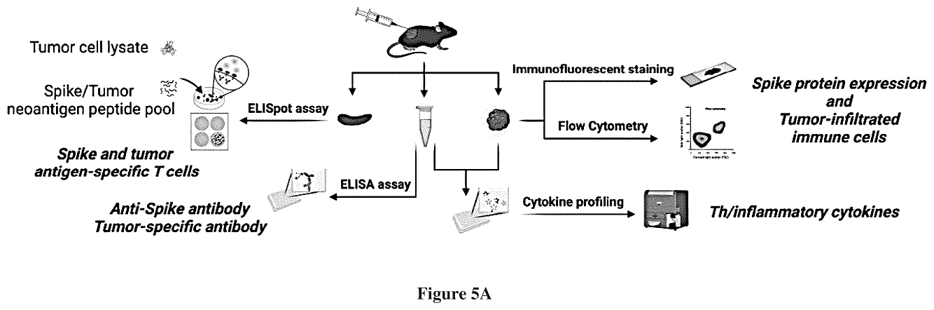

To test whether BNT16262 can be re-purposed as a cancer therapeutic drug, the therapeutic efficacy of BNT162b2 was first tested against B16F10 melanoma cancer in BNT162b2 vaccinated mice. Two treatment schemes were established. In the first scheme, mice were vaccinated with BNT16b2 before B16F10 cancer cell implantation. In the other scheme, mice were inoculated with B16F10 cancer cells before BNT162b2 vaccination. For the first scheme, the mice were administered with two doses of BNT162b2 vaccine 21 days apart by intramuscular injection and inoculated 3×10⁵ B16F10 tumor subcutaneously. Then, intratumoral injections of BNT162b2 were performed for the treatment group and PBS for the control group with equal volume on the 5th day, 10th day, and 17th day post tumor inoculation (FIG. 3A). During the treatment, the tumor volumes were measured every three days (FIGS. 3B, 3C, and 3D). On Day 20, compared to the PBS i.t. group, the tumor growth was significantly inhibited in the BNT i.t. group (FIGS. 3B, 3C, 3D, 3E). For the second scheme, 2×10⁵ B16F10 cancer cells were first inoculated subcutaneously. After that, intramuscular vaccination of BNT162b2 was performed on Day 2 and Day 5 post tumor inoculation, then intratumoral injection of BNT162b2 was performed on Day 10, Day 15, and Day 20 post tumor inoculation (FIG. 4A). The tumor volumes were measured every two days (FIGS. 4B, 4C, 4D). On Day 23, mice were sacrificed. The kinetics of tumor growth shown in the BNT i.t group indicated the tumor growth was significantly suppressed compared to that in the PBS i.t. group (FIGS. 4B, 4C, 4D, 4E). Besides, the therapeutic efficacy was tested in the MB49 subcutaneous model (FIG. 4F), 4T1 breast orthotopic model (FIG. 4K), and CT26 subcutaneous model (FIG. 4R) as well. and the tumor growth was monitored and measured every two days, The result showed that the BNT162b2 therapy also can effectively inhibit the MB49 bladder cancer tumor growth (FIGS. 4G, 4H, 4I, 4J), 4T1 breast cancer tumor growth (FIGS. 4L, 4M, 4N, 4O), and CT26 colon cancer tumor growth (FIGS. 4S, 4T, 4U, 4V). In addition to the therapeutic efficacy, the results also demonstrated that the BNT162b2 therapy also can inhibit breast cancer lung metastasis in the 4T1 orthotopic model (FIGS. 4P, 4Q).

### 2) BNT162b2 Cancer Therapy Induces Potent Anti-Tumor Specific T Cell Immune Responses.

To test the specific anti-tumor immune responses, the tumor, spleen, and serum samples after BNT162b2 cancer vaccine therapy in the B16F10 model were collected (FIG. 5A). The expression of spike protein in the tumor tissue after intratumoral injection of BNT162b2 was first detected. Immunofluorescence staining of spike protein on tumor sections showed that the SARS-COV-2 spike protein is expressed on the surface and inside of the tumor cells in BNT162b2 injected tumor at a very high level (FIGS. 5D, 5E), indicating that the invention have successfully forced the tumor cells to express the spike protein as an artificial ‘tumor-specific antigen’ in high-level post BNT162b2 administration.

Next, the results showed the BNT162b2 therapy recruited abundant T cells infiltrated into the tumor (FIGS. 5B, 5C, 5F). It was then measured whether the memory immune responses induced by previous BNT162b2 vaccination could induce potent immune responses against the tumor cells. To detect anti-spike and other anti-tumor T cell responses, the splenocytes post all vaccination and treatment steps were collected and then ELISpots assay were performed with the spike protein peptide pool or B16F10 cell lysate from cells untreated with BNT162b2 as a stimulator, respectively. The result demonstrated potent anti-spike protein antibodies (FIG. 5H) and T-cell activity (FIGS. 5I, 5J) against the spike protein of SARS-CoV-2. Furthermore, specific T cell responses against the B16F10 cell lysate with no spike protein can be detected in the mice with BNT162b2 intratumoral injection group but not the PBS-treatment group, indicating that extensive tumor-antigen spreading was induced by intratumoral injection of BNT162b2 vaccine. This tumor-antigen spreading also was validated in the MB49 model by MB49 neoantigens stimulation (FIGS. 10A, 10B). Interestingly, it was also detected T cell responses targeting liver cancer cell ML-1, which is unrelated to the B16F10 cell, indicating that BNT162b2-induced antigen spreading provoked anti-tumor T cell responses against some common tumor antigens as well (FIGS. 5I, 5J).

In addition to the T cell responses, the role of B cell and antibody responses in this BNT16262 cancer therapy were investigated as well. For studying B cell-related responses, BNT162b2 cancer therapy was tested in the μMT mouse, which lacks mature B lymphocytes [27]. The B16F10 melanoma subcutaneous model was established in this μMT mice and performed vaccine therapy as the second scheme of B16F10 melanoma treatment (FIG. 5K). The result showed that BNT162b2 therapeutic efficacy of the B16F10 melanoma model in μMT mice is as good as in the wild type mice (FIGS. 5L to 5O). However, the therapeutic efficacy cannot be detected in the Rag14 mice, which do not produce mature B and T lymphocytes (FIGS. 5L to 5O). These results indicated that BNT162b2 cancer therapy is independent of mature B cells but it's T cell dependent.

Furthermore, the splenocytes from the mice that got twice BNT162b2 vaccination or without BNT162b2 vaccination were incubated with the BNT162b2 transfected B16F10, after 20 hours of incubation, we detected the rapid activation of T cells by flow cytometry (Figure SP). Flow cytometry results showed after incubation, BNT162b2 transfected B16F10 can rapidly activate the T cells by upregulating the expression of CD69 and increasing the secretion of IFN-γ (FIGS. 5Q, 5R), the result suggested the memory T cells induced by BNT162b2 can be rapidly activated by recontacting Spike protein-expressing tumor cells.

These results indicated BNT162b2 cancer therapy mainly relies on T cell responses. and it can induce rapid T cell activation to target the tumor cells with Spike protein expression by BNT162b2 transfection, which finally provokes the first attack of tumor cells, then induces more tumor antigens release to induce more extensive tumor-specific immune responses by antigen spreading.

### 3) Systemic and Intratumoral Immune Activation can be Induced by BNT162b2 Cancer Therapy.

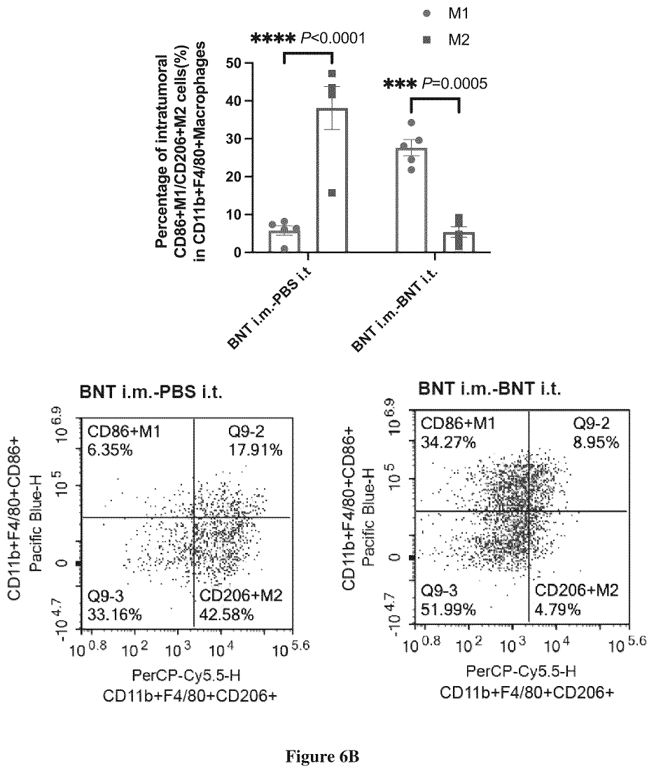

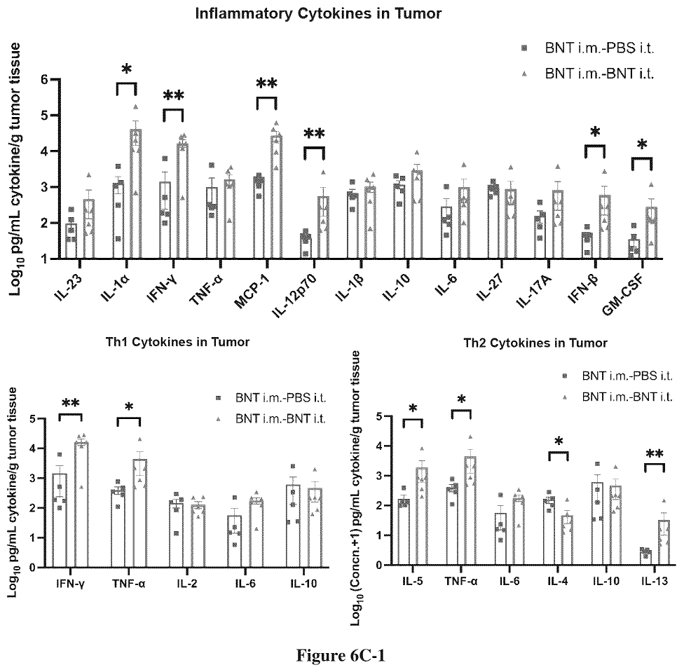

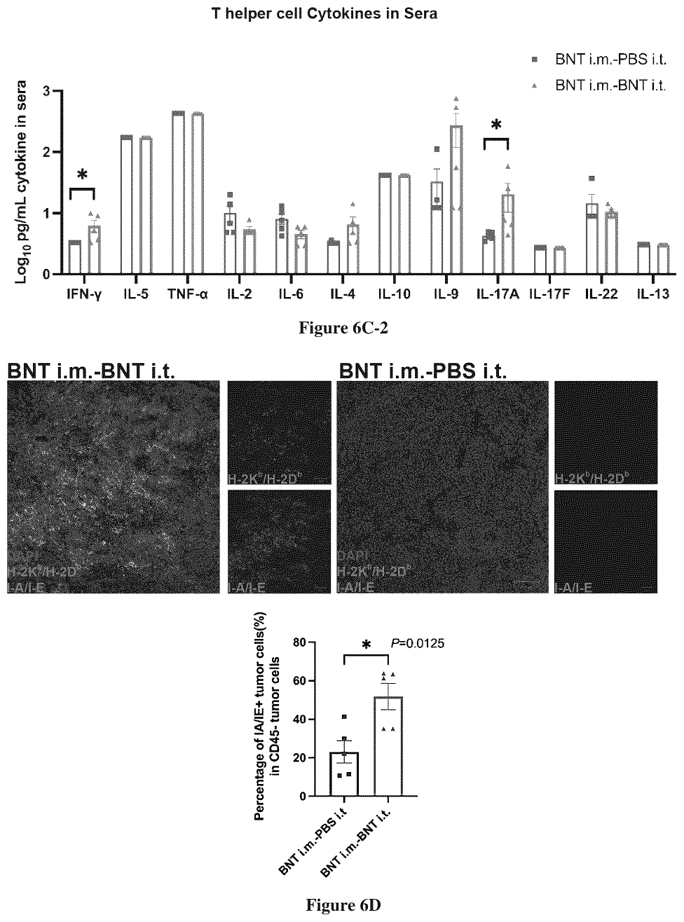

To further test whether BNT162b2 therapy can activated intratumoral immune cells and systemic immunity to provoke potent anti-tumor immune responses. Firstly, the intratumoral immune cells were tested by immunofluorescent staining and flow cytometry (FIGS. 5G-1, 5G-2, 5G-3 and 5G-4). These results showed the BNT162b2 therapy induced more intratumoral immune cells recruitment, including T cells, NK cells, Antigen Presenting Cells (APCs), Macrophages, and Neutrophils (FIGS. 5F, 5G-1, 5G-2, 5G-3 and 5G-4). Besides, BNT162b2 intratumoral injections also induced the reversion of tumor suppressive microenvironment. On the one hand, BNT162b2 therapy improved the intratumoral expression of MHC-I/MHC-II and the pro-inflammatory cytokine levels in tumors or in circulation (FIGS. 6C-1, 6C-2, D), on the other hand, BNT162b2 therapy also reversed the intratumoral macrophages from pro-tumor phenotype M2 to anti-tumor phenotype M1 (FIGS. 6A, 6B). In addition, transcriptome sequencing was conducted to study the intratumoral immune activation (FIG. 5S). It was found that the differential gene expression profile between intratumoral BNT162b2 treatment group (BNT i.m.-BNT i.t. group) and control groups (PBS i.t. group and BNT i.m.-PBS i.t. group) at different time points showed little overlap (FIG. 5T), suggesting dynamic and significant changes occurred in the TME. The Gene Ontology (GO) enrichment analysis revealed that myeloid leukocytes including neutrophils and macrophages were activated throughout the course of intratumoral BNT162b2 treatment (FIG. 5U). The results also showed a significant upregulation with a high degree of correlation in the gene expression of CD28 and B7 family members (CD80, CD86), which suggests that co-stimulatory signals from antigen-presenting cells could be involved in T cell activation (FIG. 5V).

### 4) BNT162b2 Cancer Therapy Induces Potent Tumor-Antigen Spreading by Killed Tumor Cells, More Tumor Derived Heat Shock Protein Secretion, and Tumor Antigens-Containing Exosome Secretion.

This invention has indicated that the BNT162b2 cancer therapy could induce robust tumor-antigen spreading and induce potent tumor-specific T-cell responses by killed tumor cells (FIGS. 5I, 5J).

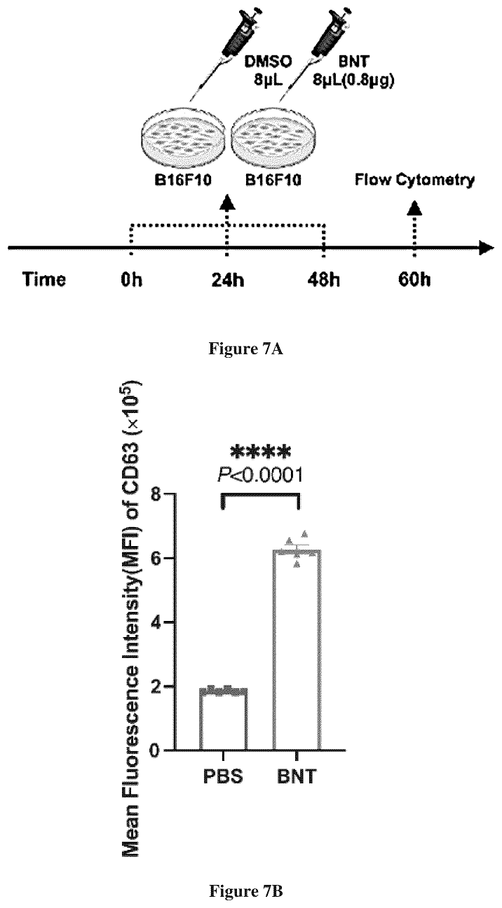

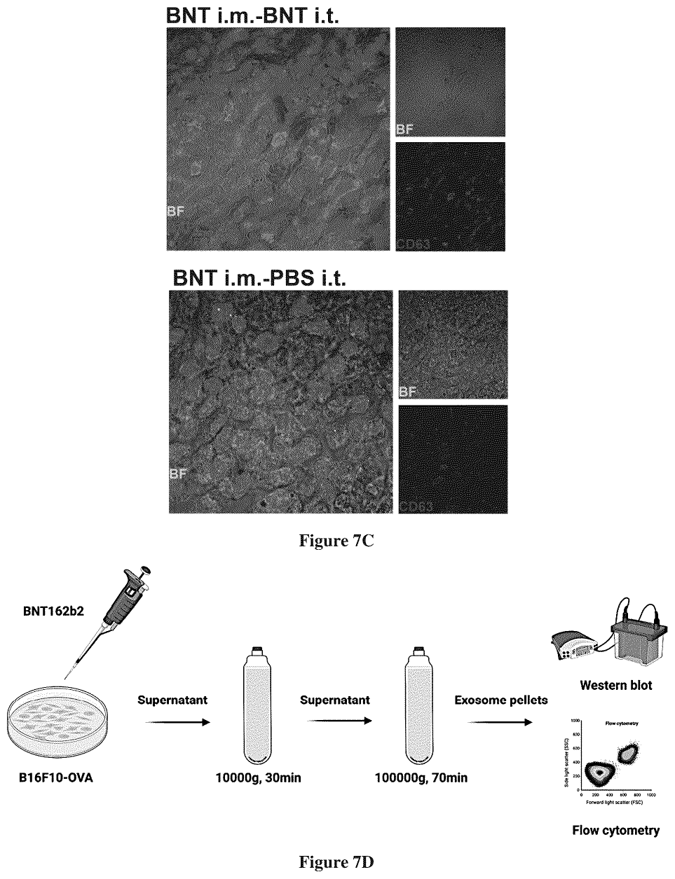

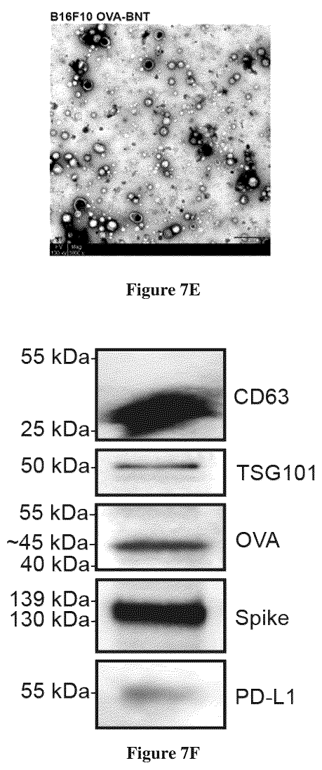

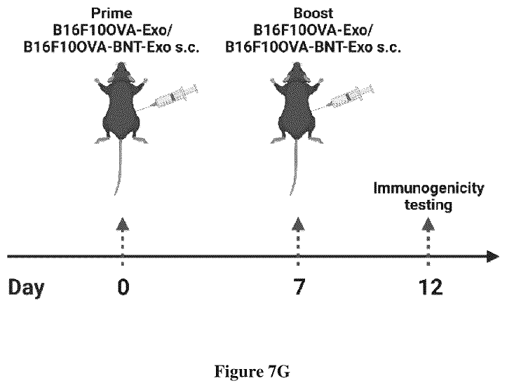

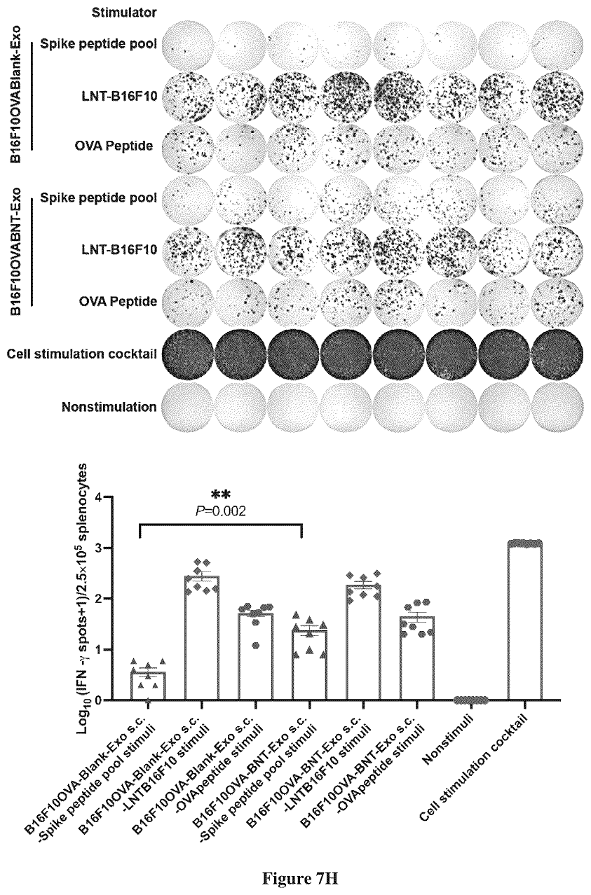

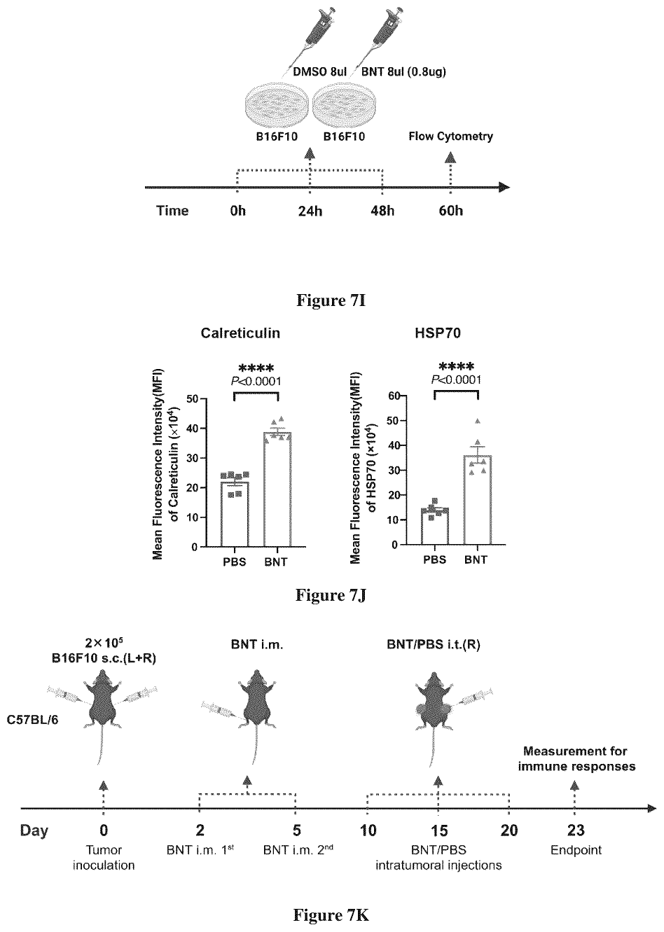

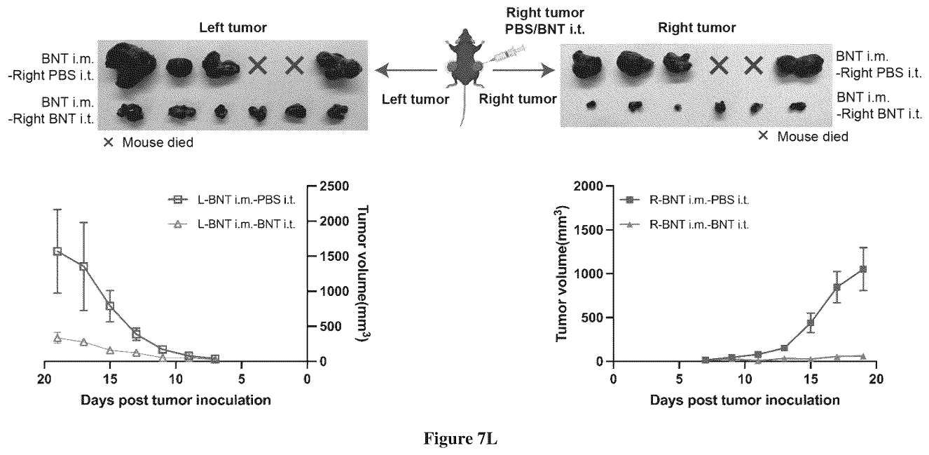

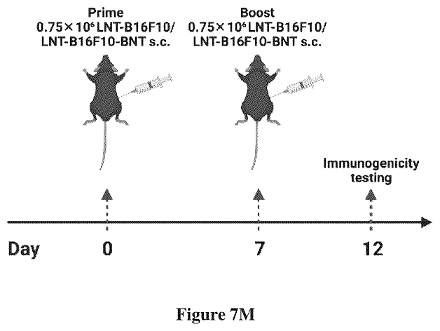

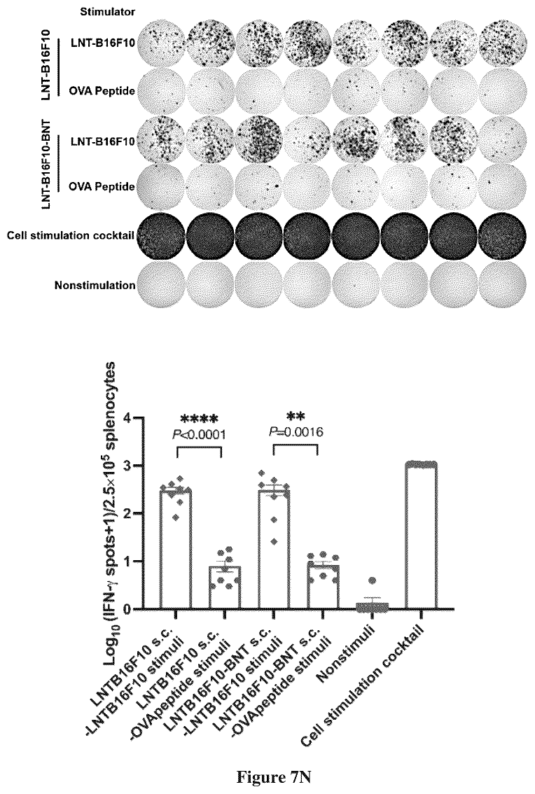

In addition, a study from another group indicated that after SARS-Cov-2 mRNA vaccination, circulating exosomes with SARS-COV-2 spike protein can be detected [29]. This observation prompted the hypothesis that intratumoral vaccination by BNT162b2 stimulates tumors to release exosomes containing not only SARS-COV-2 spike protein but also tumor-specific antigens. The release of these exosomes could induce antigen spreading and tumor-specific T cell responses, which in turn results in tumor suppression. To test whether BNT162b2 stimulates tumor antigen-containing exosome release, the B16F10-OVA tumor cell line (B16F10-OVA: B16F10 cell line with stable expression of OVA protein) was transfected with BNT162b2. The supernatant of B16F10-OVA culture medium was collected, and the total exosomes secreted by B16F10-OVA cell line were isolated. These exosomes were identified by transmission electron microscopy (TEM), flow cytometry, immunofluorescent staining, and western blot (FIGS. 7A to 7F), and the flow cytometry and immunofluorescent staining results showed the transfection of BNT162b2 induced more exosomes secretion (FIGS. 7A, 7B), and western blot and immunogenicity test results indicated these exosomes contain exosome markers, spike protein and OVA protein at the same time, and these exosomes can provoke tumor antigens and spike-specific T cell responses (FIGS. 7F to 7H). These results provide a possible mechanism for tumor-antigen spreading induced by BNT162b2 cancer therapy.

Furthermore, studies from other groups showed the Intracellular Heat Shock Protein (HSP) act as chaperones of tumor antigens-derived peptides and have been implicated in the transfer and presentation of tumor antigens [30], the data of this invention also suggested an increased expression level of heat shock proteins (HSPs), Calreticulin and HSP70, in the tumor cells post BNT162b2 treatments (FIGS. 7I, 7J).

For further validate the antigen spreading and systemic anti-tumor immune responses can be induced by BNT162b2 treatment, the therapeutic efficacy of BNT162b2 in the bilateral tumor model was tested (FIG. 7K), the therapeutic efficacy result showed the BNT162b2 treatment not only can inhibit the growth of the tumor with treatment, but also can inhibit the growth of the other tumor without any treatment (FIG. 7L). These results suggested the BNT162b2 cancer therapy can strongly provoke the antigen spreading to induce tumor antigens-specific immune responses and also show the potent distal inhibition of tumor growth.

In addition, the antigen spreading induced by dead tumor cells was also validated (FIG. 7M), the result suggested the liquid nitrogen treated (LNT)-B16F10 can strongly induced the T cell responses against tumor cells, which also indicated a way to induce tumor antigen spreading (FIG. 7N).

### 5) The Therapeutic Efficacy of BNT162b2 Cancer Therapy can be Enhanced by Anti-PD-L1 Therapy Combination.

Although the BNT162b2 therapy can significantly inhibit tumor growth, it cannot eliminate the tumor totally. Whether combinational therapy can enhance the therapeutic efficacy of BNT162b2 in tumors was investigated. Firstly, the expression level of PD-L1 in the tumor post-BNT162b2 treatments and PBS treatments was measured (FIG. 8A). The result suggested that BNT162b2 therapy increased the PD-L1 expression in tumors compared with PBS ones (FIG. 8B). It was then found that PD-L1 expression has a significant increase in CD45+ Leukocytes but no increase in tumor cells (FIG. 8C). The transcriptome sequencing data also showed the expressions of IFNγ (FIG. 8F) and pattern recognition receptor signaling pathways (FIG. 5U) were significantly upregulated or enriched following the intratumoral BNT162b2 injections, which is similar to the mRNA vaccinology results reported by Arunachalam et al. [31]. Interestingly, a significant upregulation of CD274 (PD-L1) during the first three time points after intratumoral BNT162b2 injection was observed (FIG. 8D). It was reported that tumor-associated neutrophils and macrophages negatively regulate the adaptive immunity via the mechanism of an increased expression of PD-L1 in different tumor types [32-34]. The analysis also suggested that PD-L1 expression was strongly correlated with the activation of neutrophils and macrophages (FIG. 8E). The flow cytometry results also showed PD-L1 expression on macrophages in tumors is high with or without BNT162b2 therapy at the endpoint. Importantly, PD-L1 expression in intratumoral neutrophils showed a significant increase in BNT162b2 treatmental tumors (FIG. 8G). Furthermore, the immunofluorescent staining indicated the co-location of PD-L1 and neutrophil marker Gr-1, and the increased percentage of PD-L1 post-BNT162b2 therapy in neutrophil is not only in total intratumoral neutrophils but also in total live cells (FIG. 8H).

Although tumor-derived exosomes can prime tumor-specific immune responses as described above, many studies showed that tumor-derived exosomes also play a role in facilitating tumor growth [35-37]. Therefore, the prophylactic efficacy and the therapeutic efficacy of BNT162b2-treated tumor-derived exosomes in the B16F10 subcutaneous model were tested (FIGS. 8I, 8L). The results showed the BNT162b2-treated tumor-derived exosomes have no significant therapeutic efficacy in the prophylactic model, and even significantly facilitate the tumor growth in the therapeutic model (FIGS. 8J, SK, 8M, 8N).

Due to the increased expression of PD-L1 on the intratumoral macrophages and neutrophils, and the increased number of PD-L1-containing BNT162b2 treated tumor-derived exosomes. Whether combining the anti-PD-L1 therapy can enhance the therapeutic efficacy of BNT162b2 cancer therapy was investigated (FIG. 9A). The result indicated that anti-PD-L1 therapy can significantly improve the therapeutic efficacy of BNT162b2 (FIGS. 9B, 9C), and tumors were totally eliminated in three of the five mice with combinational therapy (FIG. 9B), these results strongly indicated the translational potential of BNT162b2 and combinational therapy of BNT162b2 and anti-PD-L1 in various kinds of cancers. Furthermore, we tested the therapeutic efficacy of BNT162b2 in advanced tumor model (FIG. 9D), the result showed the potent therapeutic efficacy in advanced tumor model, and the efficacy also can be further enhanced by combining anti-PD-L1 therapy (FIGS. 9E, 9F).

6) The Therapeutic Efficacy of Other Pathogen Antigens Encoded mRNA Vaccines.

In addition to the BNT16262 encoding the Spike protein of SARS-COV-2, the therapeutic efficacy of Spike protein from another kind of coronavirus, HKU1-CoV was investigated using the same therapeutic strategy as BNT162b2 (FIG. 11A), which showed a great therapeutic efficacy of lipid nanoparticle encapsulated HKU1 CoV-Spike protein encoded mRNA vaccine in melanoma subcutaneous model (FIGS. 11B, 11C, 11D, 11E). Besides, a great therapeutic efficacy was also showed for lipid nanoparticle encapsulated SEA protein encoded mRNA vaccine (FIG. 12A, 12B, 12C, 12D, 12E, 12G). Interestingly, due to the superantigen can activate the T cell directly by crosslinking MHC II to TCR, the introduction of superantigen into the tumor is hypothesized to induce potent T cell responses directly targeting tumor cells with superantigen expression. The vaccine therapeutic results suggested that SEA mRNA vaccine intratumoral injections alone can also lead to a comparable therapeutic efficacy to SEA mRNA vaccine intratumoral injections post SEA mRNA vaccine intramuscular vaccination (FIG. 12A, 12B, 12C, 12D, 12E, 12F, 12G). These results showed the cancer immunotherapy strategy of this invention indeed has a great therapeutic efficacy and was validated in different pathogen antigen-based mRNA vaccines.

### Example 3

### Discussion

The successful clinical application of immunotherapy helps patients with cancer prolong their lives. However, not all cancer patients benefit from current immunotherapy because of the decrease of tumor antigenicity or immunogenicity, which can be caused by tumor-antigen loss, antigen-presenting issues, and so on. In addition, the formation of a suppressive tumor microenvironment also results in anti-tumor immune response suppression, such as the intratumoral regulatory immune cells and the increased expression of immune checkpoints. On the other hand, the effective tumor immunity cycle also indicates the importance of tumor antigenicity, immunogenicity, and tumor microenvironment conversion. Therefore, for better anti-tumor immunotherapies, strategies are needed to not only induce the specific anti-tumor immune responses but also help to increase the antigenicity and immunogenicity of the tumor, at the same time, to convert the suppressive tumor microenvironment to a favorite one for immunotherapy. An effective strategy by repurposing the BNT162b2 SARS-COV-2 vaccine and other pathogen antigen-based mRNA vaccines as anti-tumor drugs were demonstrated.

The BNT162b2 or other pathogen antigen-based mRNA vaccines anti-tumor therapy was tested in different tumor models with different schemes. In the B16F10 melanoma model, intramuscular vaccination before or after tumor inoculation showed very high therapeutic efficacy. Good efficacy of BNT16262 can also be detected in 4T1 breast cancer model, CT26 colon cancer model, and MB49 bladder cancer model. Although in 4T1 model, BNT162b2 therapy was not as efficient as for the melanoma model, 4T1 cancer cell lung metastasis is strongly inhibited. In addition, it was found that BNT162b2 intratumoral injections recruited immune cells into the tumor and induce the pro-inflamatory cytokines secretion, which promote the intratumoral T cells activation. The mechanism studies also suggested the BNT162b2 anti-melanoma therapy is still working in μMT mice, but the therapeutic efficacy disappeared in Rag1^{-/-} mice, indicating this strategy relies on T cell responses, but it is mature B cell-independent. To understand the mechanisms underlying BNT162b2-induced tumor antigen spreading and tumor-specific immune response, whether spike protein and tumor antigens-containing exosomes were released after BNT162b2 treatment was investigated. It was found that BNT162b2 administration induced more exosome secretion compared with non-administrated ones. At the same time, spike protein and tumor-antigen can be detected in the exosomes isolated from the group with BNT162b2 administration. This result indicated the BNT162b2 administration helps the tumor-antigen spreading by more tumor-antigen-containing tumor-derived exosomes secretion, then these tumor-antigen-containing exosomes can induce tumor-antigen specific immune responses which finally result in a more efficient anti-tumor responses activation. In addition to the tumor-derived exosomes, more dead tumor cells in the BNT162b2 treated tumor also can induce more tumor antigens-specific responses activation. The antigen spreading in the bilatral tumor model was also validated, and found the BNT162b2 cancer therapy also can inhibit the distal and non-treated tumor growth, indicating the systemic tumor-specific immune responses activation.

Although the BNT162b2 strongly inhibited the tumor growth in the tumor-bearing mice, it cannot totally eliminate the tumors, so the tumor microenvironment changes post the BNT162b2 therapy was investigated. The data showed, BNT162b2 Intratumoral injections induced high expression level of PD-L1 in the Leukocytes, especially in intratumoral macrophages and neutrophils. These result indicated the potential of combinational therapy of anti-PD-L1. The therapeutic test of combinational therapy of BNT162b2 and anti-PD-L1 indeed showed a potent cancer therapeutic efficacy, interestingly, in some cases, tumors can be totally eliminated post combinational therapy.

In addition to the BNT162b2, encoding SARS-COV-2 Spike protein, other pathogen antigen-based mRNA vaccines for cancer therapy, such as HKU1 CoV-Spike protein encoded mRNA vaccine and SEA protein encoded mRNA vaccine, were also developed which also showed great therapeutic efficacies for cancer therapy.

Due to the COVID-19 pandemic, governments around the world are giving vaccination to their citizens urgently to prevent the virus from large-scale spreading. To date, more than 70% population all over the world has been fully vaccinated, and the vaccinated or infected people have memory immunity against spike protein. Besides, widely vaccinations or common coronavirus and bacterial infections also provoked memory immunity against other viral or bacterial antigens, which provide the potential of cancer therapy by using these pathogen antigen-based mRNA vaccines. Additionally, compared to conventional cancer vaccine design, this strategy of cancer therapy need not analyze the tumor mutation of cancer patients individually, and the expression of spike protein in the tumor can be regarded as a common tumor-specific antigen. These advantages make this cancer therapy strategy by pathogen antigen-based mRNA vaccine more universal and more convenient for clinical application. On the other hand, BNT162b2 and other SARS-COV-2 mRNA vaccines have been approved for clinical application, and the widespread vaccination is sufficient to prove their efficiency and safety. Because of these, BNT162b2 can be quickly applicated in the clinic for cancer therapy by expressing the spike protein intratumorally. The expression of the spike protein will induce the activation of anti-spike memory immunity and tumor-specific immune responses rapidly to attack the tumor. More importantly, those other foreign antigens from pathogens also have the potential to be the off-the-shelf vaccine for various cancer therapy as validated in this invention, especially the infectious diseases people have got vaccinated to prevent. In conclusion, this invention not only provides an exciting application potential to repurpose the COVID-19 mRNA vaccine for cancer immunotherapy with robust therapeutic efficacy but also proposes one new cancer therapy strategy by awakening and redirecting the memory immunity from infections or vaccinations for cancer therapy. It is believed that these therapy strategies can provide patients more choices and hopes.

While the detail of this invention has been described above, it should be noted that the invention is not limited thereto but can be practiced in various ways as illustrated in the following claims.

### REFERENCES

1. Chen, D. S. and I. Mellman, Oncology meets immunology: the cancer-immunity cycle. Immunity, 2013. 39 (1): p. 1-10.
2. Aktar, N., et al., Understanding of Immune Escape Mechanisms and Advances in Cancer Immunotherapy. J Oncol, 2022. 2022: p. 8901326.
3. Beatty. G. L. and W. L. Gladney, Immune escape mechanisms as a guide for cancer immunotherapy. Clin Cancer Res, 2015. 21 (4): p. 687-92.
4. Newman, J. H., et al., Intratumoral injection of the seasonal flu shot converts immunologically cold tumors to hot and serves as an immunotherapy for cancer. Proc Natl Acad Sci USA, 2020. 117 (2): p. 1119-1128.
5. Russell, S. J., et al., Advances in oncolytic virotherapy. Commun Med (Lond), 2022. 2: p. 33.
6. Russell, S. J., K. W. Peng, and J. C. Bell, Oncolytic virotherapy. Nat Biotechnol, 2012. 30 (7): p. 658-70.
7. Yu, B., et al., Explicit hypoxia targeting with tumor suppression by creating an “obligate” anaerobic Salmonella Typhimurium strain. Sci Rep, 2012. 2: p. 436.
8. Ning, B. T., et al., Treatment of Neuroblastoma with an Engineered “Obligate” Anaerobic Salmonella typhimurium Strain YB1. J Cancer, 2017. 8 (9): p. 1609-1618.
9. Lin, Q., et al., IFN-gamma-dependent NK cell activation is essential to metastasis suppression by engineered Salmonella. Nat Commun, 2021. 12 (1): p. 2537.
10. DeBruyne, L. A., et al., Direct transfer of a foreign MHC gene into human melanoma alters T cell receptor V beta usage by tumor-infiltrating lymphocytes. Cancer Immunol Immunother, 1996. 43 (1): p. 49-58.
11. GREGORY E. PLAUTZ*t, Z.-Y. Y., BEI-YUE WU*, XIANG GAOt.LEAF HUANG*, AND GARY J.NABEL*§. Immunotherapy of malignancy by in vivo gene transfer into tumors (genetherapy/majorhistocompatibilitycomplex/cancer/Tcells/adenocarcinoma). Proc. Natl. Acad. Sci. USA, 1993. 90: p. 4645-4649.
12. Schmidt, W., et al., Transloading of tumor cells with foreign major histocompatibility complex class I peptide ligand: a novel general strategy for the generation of potent cancer vaccines. Proc Natl Acad Sci USA, 1996. 93 (18): p. 9759-63.
13. El-Khoueiry, A. B., et al., Nivolumab in patients with advanced hepatocellular carcinoma (CheckMate 040): an open-label, non-comparative, phase 1/2 dose escalation and expansion trial. Lancet, 2017. 389 (10088): p. 2492-2502.
14. Brahmer, J. R., et al., Safety and activity of anti-PD-L1 antibody in patients with advanced cancer. N Engl J Med, 2012. 366 (26): p. 2455-65.
15. Lui, D. T. W., et al., Thyroid Immune-Related Adverse Events in Patients with Cancer Treated with anti-PD1/anti-CTLA4 Immune Checkpoint Inhibitor Combination: Clinical Course and Outcomes. Endocr Pract, 2021. 27 (9): p. 886-893.
16. Yau, T., et al., Efficacy and Safety of Nivolumab Plus Ipilimumab in Patients With Advanced Hepatocellular Carcinoma Previously Treated With Sorafenib: The CheckMate 040 Randomized Clinical Trial. JAMA Oncol, 2020. 6 (11): p. e204564.
17. Rafiq, S., C. S. Hackett, and R. J. Brentjens, Engineering strategies to overcome the current roadblocks in CAR T cell therapy. Nat Rev Clin Oncol, 2020. 17 (3): p. 147-167.
18. Labanieh. L., R. G. Majzner, and C. L. Mackall, Programming CAR-T cells to kill cancer. Nat Biomed Eng, 2018. 2 (6): p. 377-391.
19. Sterner, R. C. and R. M. Sterner, CAR-T cell therapy: current limitations and potential strategies. Blood Cancer J, 2021. 11 (4): p. 69.
20. June, C. H., et al., CAR T cell immunotherapy for human cancer. Science, 2018. 359 (6382): p. 1361-1365.
21. Wagner, J., et al., CAR T Cell Therapy for Solid Tumors: Bright Future or Dark Reality? Mol Ther. 2020. 28 (11): p. 2320-2339.
22. Organization, W. H. Background document on the mRNA vaccine BNT16262 (Pfizer-BioNTech) COVID-19. 2021 Available against from:

https://www.who.int/publications/i/item/background-document-on-mrna-vaccine-bnt 162b2-(pfizer-biontech)-against-covid-19.
23. Polack, F. P., et al., Safety and Efficacy of the BNT16262 mRNA Covid-19 Vaccine. N Engl J Med, 2020. 383 (27): p. 2603-2615.
24. Sahin. U., et al., BNT162b2 vaccine induces neutralizing antibodies and poly-specific T cells in humans. Nature, 2021. 595 (7868): p. 572-577.
25. Li, C., et al., Mechanisms of innate and adaptive immunity to the Pfizer-BioNTech BNT162b2 vaccine. Nat Immunol, 2022. 23 (4): p. 543-555.
26. Hannah Ritchie, E. M., Lucas Rodés-Guirao, Cameron Appel, Charlie Giattino, Esteban Ortiz-Ospina, Joe Hasell, Bobbie Macdonald, Diana Beltekian and Max Roser. Coronavirus Pandemic (COVID-19). 2020; Available from: https://ourworldindata.org/coronavirus.
27. Kitamura, D., et al., A B cell-deficient mouse by targeted disruption of the membrane exon of the immunoglobulin mu chain gene. Nature, 1991. 350 (6317): p. 423-6.
28. Mombaerts P, Iacomini J, Johnson R S, Herrup K, Tonegawa S, Papaioannou V E., RAG-1-deficient mice have no mature B and T lymphocytes. Cell, 1992. 68(5):869-77.
29. Bansal, S., et al., Cutting Edge: Circulating Exosomes with COVID Spike Protein Are Induced by BNT162b2 (Pfizer-BioNTech) Vaccination prior to Development of Antibodies: A Novel Mechanism for Immune Activation by mRNA Vaccines. J Immunol, 2021. 207 (10): p. 2405-2410.
30. Binder, R. J., Immunosurveillance of cancer and the heat shock protein-CD91 pathway. Cell Immunol, 2019. 343: p. 103814.
31. Arunachalam, P. S., et al., Systems vaccinology of the BNT162b2 mRNA vaccine in humans. Nature, 2021. 596 (7872): p. 410-416.
32. He, G., et al., Peritumoural neutrophils negatively regulate adaptive immunity via the PD-L1/PD-1 signalling pathway in hepatocellular carcinoma. J Exp Clin Cancer Res, 2015. 34: p. 141.
33. Wang, T. T., et al., Tumour-activated neutrophils in gastric cancer foster immune suppression and disease progression through GM-CSF-PD-L1 pathway. Gut, 2017. 66 (11): p. 1900-1911.
34. Liu, Y., et al., Immune Cell PD-L1 Colocalizes with Macrophages and Is Associated with Outcome in PD-1 Pathway Blockade Therapy. Clin Cancer Res, 2020. 26 (4): p. 970-977.
35. Li, Y., et al., Targeted inhibition of tumor-derived exosomes as a novel therapeutic option for cancer. Exp Mol Med, 2022. 54 (9): p. 1379-1389.
36. TL., W., Tumor-Derived Exosomes and Their Role in Cancer Progression. Adv Clin Chem., 2016. 74:103-41.
37. Tang, Q., et al., Tumor-derived exosomes in the cancer immune microenvironment and cancer immunotherapy. Cancer Lett, 2022. 548: p. 215823.

What is claimed is:

1. A method to increase antigenicity or immunogenicity of a tumor in a subject, said method comprises the step of delivering lipid nanoparticles loaded with mRNA sequences encoding one or more pathogen antigen proteins into said tumor.

2. The method of claim 1, wherein said one or more pathogen antigen proteins is an antigen targeted in a vaccine previously administered to or to be administered to said subject.

3. The method of claim 1, wherein said one or more pathogen antigen proteins is selected from the group consisting of coronavirus proteins, human papillomavirus proteins, respiratory syncytial virus, human immunodeficiency virus proteins, hepatitis virus and influenza virus proteins.

4. The method of claim 2, wherein said vaccine is selected from the group consisting of BioNTech BNT162b2, BioNTech vaccine COVID-19 Omicron-modified Bivalent vaccine, and Moderna vaccine: mRNA-1273.

5. The method of claim 1, wherein said lipid nanoparticles are delivered by an intratumoral injection.

6. The method of claim 5, wherein said intratumoral injection is an mRNA vaccine.

7. The method of claim 6, wherein said mRNA vaccine is an mRNA vaccine directed against one or more antigen selected from the group consisting of bacterial antigen, bacterial superantigen, viral antigen and viral superantigen.

8. The method of claim 1, wherein said method further comprises the step of immunizing said subject against said one or more pathogen antigen proteins or their variants.

9. The method of claim 1, wherein said subject has been previously immunized against said one or more pathogen antigen proteins or their variants.

10. The method of claim 1, wherein said tumor is selected from the group consisting of melanoma, breast cancer, bladder cancer, colon cancer, gastric cancer, pancreatic cancer, blood cancer, lung cancer, and liver cancer.

11. A method to treat a subject having one or more tumors, said method comprises increasing antigenicity or immunogenicity of at least one tumor among said one or more tumors using the method of claim 1.

12. The method of claim 11, wherein said at least one tumor is a primary tumor or secondary tumor.

13. The method of claim 11, wherein said method further comprises co-administering an immune checkpoint inhibitor to said subject.

14. The method of claim 13, wherein said immune checkpoint inhibitor is one or more selected from the group consisting of anti-PD-1, anti-CTLA4, and anti-PD-L1.

15. An intratumoral injection for treating a tumor in a subject, said intratumoral injection comprises lipid nanoparticles loaded with mRNA encoding one or more pathogen antigen proteins.

16. The intratumoral injection of claim 15, wherein said one or more pathogen antigen proteins is an antigen targeted in a vaccine previously administered to or to be administered to said subject.

17. The intratumoral injection of claim 15, wherein said tumor is selected from the group consisting of melanoma, breast cancer, bladder cancer, colon cancer, gastric cancer, pancreatic cancer, blood cancer, lung cancer, and liver cancer.

18. A kit for treating tumor in a subject, comprising the intratumoral injection of claim 15.

19. The kit of claim 18, wherein said kit further comprises an immune checkpoint inhibitor.

20. The kit of claim 18, wherein said kit further comprises a vaccine for inoculating said subject against said one or more pathogen antigen proteins.
